(19) United States
(12) Patent Application Publication
(10) Pub. No.: US 2012/0270929 A1
(43) Pub. Date: Oct. 25, 2012

(54) MODULATION OF TTC39 EXPRESSION TO INCREASE HDL

(75) Inventors: Rosanne M. Crooke, Carlsbad, CA (US); Mark Graham, San Clemente, CA (US); Thomas A. Bell, Encinitas, CA (US)

(73) Assignee: ISIS Pharmaceuticals, Inc., Carlsbad, CA (US)

(21) Appl. No.: 13/498,055

(22) PCT Filed: Sep. 24, 2010
(86) PCT No.: PCT/US10/50292

§ 371 Date: Jul. 6, 2012

Related U.S. Application Data
(60) Provisional application No. 61/246,068, filed on Sep. 25, 2009.
(60) Provisional application No. 61/246,474, filed on Sep. 28, 2009.
(60) Provisional application No. 61/334,745, filed on May 14, 2010.

(51) Int. Cl. A61K 31/7088; A61P 1/16; A61P 3/10; A61P 3/06; A61P 9/12; A61P 9/00

(52) U.S. Cl. 514/44 R

(57) ABSTRACT

Provided herein are methods, compounds, and compositions for reducing expression of a TTC39 mRNA and protein in an animal. Also provided herein are methods, compounds, and compositions for increasing HDL and/or decreasing PCSK9 in an animal. Such methods, compounds, and compositions are useful to treat, prevent, delay, or ameliorate cardiovascular disease, or a symptom thereof.

### SEQUENCE LISTING

The present application is being filed along with a Sequence Listing in electronic format. The Sequence Listing is provided as a file entitled BIOL0115WOSEQ.txt created Sep. 24, 2010, which is approximately 568 Kb in size. The information in the electronic format of the sequence listing is incorporated herein by reference in its entirety.

### FIELD OF THE INVENTION

Provided herein are methods, compounds, and compositions for reducing expression of tetratricopeptide repeat domain 39 (TTC39) mRNA and protein in an animal. Also, provided herein are methods, compounds, and compositions comprising a TTC39 inhibitor for increasing HDL levels in an animal. Such methods, compounds, and compositions are useful, for example, to treat, prevent, or ameliorate cardiovascular disease in an animal.

### BACKGROUND

TTC39 proteins are members of the TPR (tetratricopeptide repeat) protein family. There are 3 known human isoforms of the protein: TTC39A, TTC39B and TTC39C. The TTC39 proteins have at least one TPR motif consisting of two antiparallel α-helices and tandem arrays of TPR motifs create a grooved domain capable of facilitating a wide array of protein-protein interactions (Blatch G L, Lassie M. The tetratricopeptide repeat: a structural motif mediating protein-protein interactions. BioEssays 1999; 21:932-939). Experimental evidence has shown that TPR proteins are involved in four major types of complexes: 1) molecular chaperone, 2) anaphase promotional, 3) transcriptional repression, and 4) protein transport complexes (Blatch G L, Lassie M. The tetratricopeptide repeat: a structural motif mediating protein-protein interactions. BioEssays 1999; 21:932-939; Smith D F. Tetratricopeptide repeat cochaperones in steroid receptor complexes. Cell Stress and Chaperones 2004; 9(2):109-121).

Recently during a Genome-Wide Association Study (GWAS) the TPR protein, TTC39B, was implicated as having an association with cardiovascular disease (Kathiresan S. et al. Common variants at 30 loci contribute to polygenic dyslipidemia. Nature Genetics 2009; 41(1):56-65). TTC39B expression was negatively associated with plasma high density lipoprotein (HDL) cholesterol levels and an allele associated with lower TTC39B transcript levels was also associated with higher HDL cholesterol levels. Cellular cholesterol efflux is mediated by HDL. Low levels of HDL cholesterol can be a significant predictor of atherosclerotic cardiovascular events. Plasma levels of HDL are inversely correlated with the risk of cardiovascular disease (Caveliar et al, Biochim Biophys Acta. 2006 1761: 655-66).

GWAS has implicated a number of genes in cardiovascular disease or associated genes with markers (such as cholesterol levels) for cardiovascular disease. Although TTC39B has been implicated by GWAS in cardiovascular disease, more study is required to determine the nature of the association and whether modulation of a gene product has therapeutic relevance.

The function of the TTC39 proteins has not yet been fully elucidated.

### SUMMARY OF THE INVENTION

Provided herein are methods, compounds, and compositions for inhibiting expression of TTC39 and treating, preventing, delaying or ameliorating a TTC39 related disease and/or a symptom thereof.

Certain embodiments provide a method of reducing a TTC39 isoform expression in an animal comprising administering to the animal a compound comprising a modified oligonucleotide 12 to 30 linked nucleosides in length targeted to the TTC39 isoform.

Certain embodiments provide a method of increasing HDL level in an animal comprising administering to the animal a compound comprising a modified oligonucleotide 12 to 30 linked nucleosides in length targeted to a TTC39 isoform, wherein the modified oligonucleotide reduces the TTC39 isoform expression in the animal, thereby increasing the HDL level in the animal.

Certain embodiments provide a method for treating an animal with cardiovascular disease comprising: a) identifying said animal with cardiovascular disease, and b) administering to said animal a therapeutically effective amount of a compound comprising a modified oligonucleotide 12 to 30 linked nucleosides in length targeted to a TTC39 isoform.

In certain embodiments, the TTC39 isoform is TTC39B with a sequence as set forth in GenBank Accession No. NM_{—}152574.1 (incorporated herein as SEQ ID NO: 1)

In certain embodiments, inhibition of TTC39B expression increases HDL in an animal.

### DETAILED DESCRIPTION OF THE INVENTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### DEFINITIONS

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques can be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout in the disclosure herein are incorporated by reference for the portions of the document discussed herein, as well as in their entirety.

Unless otherwise indicated, the following terms have the following meanings:

“2′-O-methoxyethyl” (also 2′-MOE and 2′-O(CH₂)₂—OCH₃) refers to an O-methoxy-ethyl modification of the 2′ position of a furosyl ring. A 2′-O-methoxyethyl modified sugar is a modified sugar.

“2′-O-methoxyethyl nucleotide” means a nucleotide comprising a 2′-O-methoxyethyl modified sugar moiety.

“5-methylcytosine” means a cytosine modified with a methyl group attached to the 5′ position. A 5-methylcytosine is a modified nucleobase.

“Active pharmaceutical agent” means the substance or substances in a pharmaceutical composition that provide a therapeutic benefit when administered to an individual. For example, in certain embodiments an antisense oligonucleotide targeted to TTC39B is an active pharmaceutical agent.

“Active target region” or “target region” means a region to which one or more active antisense compounds is targeted. “Active antisense compounds” means antisense compounds that reduce target nucleic acid levels or protein levels.

“Administered concomitantly” refers to the co-administration of two agents in any manner in which the pharmacological effects of both are manifest in the patient at the same time. Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

“Administering” means providing an agent to an animal, and includes, but is not limited to, administering by a medical professional and self-administering.

“Agent” means an active substance that can provide a therapeutic benefit when administered to an animal. “First Agent” means a therapeutic compound of the invention. For example, a first agent can be an antisense oligonucleotide targeting TTC39B. “Second agent” means a second therapeutic compound of the invention (e.g. a second antisense oligonucleotide targeting TTC39B) and/or a non-TTC39 therapeutic compound (e.g., statins, ezetamibe, niacin, fibrates, beta blockers, antithrombotics and antihypertensives).

“Amelioration” refers to a lessening of at least one indicator, sign, or symptom of an associated disease, disorder, or condition. The severity of indicators can be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding.

“Antisense inhibition” means reduction of target nucleic acid levels or target protein levels in the presence of an antisense compound complementary to a target nucleic acid compared to target nucleic acid levels or target protein levels in the absence of the antisense compound.

“Antisense oligonucleotide” means a single-stranded oligonucleotide having a nucleobase sequence that permits hybridization to a corresponding region or segment of a target nucleic acid.

“Bicyclic sugar” means a furosyl ring modified by the bridging of two non-geminal ring atoms. A bicyclic sugar is a modified sugar.

“Bicyclic nucleic acid” or “BNA” refers to a nucleoside or nucleotide wherein the furanose portion of the nucleoside or nucleotide includes a bridge connecting two carbon atoms on the furanose ring, thereby forming a bicyclic ring system.

“Cap structure” or “terminal cap moiety” means chemical modifications, which have been incorporated at either terminus of an antisense compound.

“Cardiovascular Disease” means a disease or condition affecting the heart or blood vessel of an animal. Examples of cardiovascular disease include, but are not limited to, arteriosclerosis, atherosclerosis, coronary heart disease, heart failure, hypertension, dyslipidemia, hypercholesterolemia, acute coronary syndrome, type II diabetes, type II diabetes with dyslipidemia, hepatic steatosis, non-alcoholic steatohepatitis, non-alcoholic fatty liver disease, hypertriglyceridemia, hyperfattyacidemia, hyperlipidemia and metabolic syndrome.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Chimeric antisense compound” means an antisense compound that has at least two chemically distinct regions.

“Co-administration” means administration of two or more agents to an individual. The two or more agents can be in a single pharmaceutical composition, or can be in separate pharmaceutical compositions. Each of the two or more agents can be administered through the same or different routes of administration. Co-administration encompasses parallel or sequential administration.

“Complementarity” means the capacity for pairing between nucleobases of a first nucleic acid and a second nucleic acid.

“Contiguous nucleobases” means nucleobases immediately adjacent to each other.

“Diluent” means an ingredient in a composition that lacks pharmacological activity, but is pharmaceutically necessary or desirable. For example, the diluent in an injected composition can be a liquid, e.g. saline solution.

“Dose” means a specified quantity of a pharmaceutical agent provided in a single administration, or in a specified time period. In certain embodiments, a dose can be administered in one, two, or more boluses, tablets, or injections. For example, in certain embodiments where subcutaneous administration is desired, the desired dose requires a volume not easily accommodated by a single injection, therefore, two or more injections can be used to achieve the desired dose. In certain embodiments, the pharmaceutical agent is administered by infusion over an extended period of time or continuously. Doses can be stated as the amount of pharmaceutical agent per hour, day, week, or month.

“Effective amount” or “therapeutically effective amount” means the amount of active pharmaceutical agent sufficient to effectuate a desired physiological outcome in an individual in need of the agent. The effective amount can vary among individuals depending on the health and physical condition of the individual to be treated, the taxonomic group of the individuals to be treated, the formulation of the composition, assessment of the individual's medical condition, and other relevant factors.

“Fully complementary” or “100% complementary” means each nucleobase of a nucleobase sequence of a first nucleic acid has a complementary nucleobase in a second nucleobase sequence of a second nucleic acid. In certain embodiments, a first nucleic acid is an antisense compound and a target nucleic acid is a second nucleic acid.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region can be referred to as a “gap segment” and the external regions can be referred to as “wing segments.”

“Gap-widened” means a chimeric antisense compound having a gap segment of 12 or more contiguous 2′-deoxyribonucleosides positioned between and immediately adjacent to 5′ and 3′ wing segments having from one to six nucleosides.

“HDL” means high density lipoprotein particles. Concentration of HDL in serum (or plasma) is typically quantified in mg/dL or nmol/L. “Serum HDL” and “plasma HDL” mean HDL in the serum and plasma, respectively.

“Hybridization” means the annealing of complementary nucleic acid molecules. In certain embodiments, complementary nucleic acid molecules include an antisense compound and a target nucleic acid.

“Identifying an animal with cardiovascular disease” means identifying an animal having been diagnosed with a cardiovascular disease, disorder or condition or identifying an animal predisposed to develop a cardiovascular disease, disorder or condition. For example, individuals with a familial history of dyslipidemia or hyperlipidemia can be predisposed to cardiovascular disease, disorder or condition. Such identification can be accomplished by any method including evaluating an individual's medical history and standard clinical tests or assessments.

“Immediately adjacent” means there are no intervening elements between the immediately adjacent elements.

“Individual” means a human or non-human animal selected for treatment or therapy.

“Internucleoside linkage” refers to the chemical bond between nucleosides.

“Isoforms” means different genes with similar functional domains or sequence homology in some domains.

“Linked nucleosides” means adjacent nucleosides which are bonded together.

“Mismatch” or “non-complementary nucleobase” refers to the case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e. a phosphodiester internucleoside bond).

“Modified nucleobase” refers to any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

“Modified nucleotide” means a nucleotide having, independently, a modified sugar moiety, modified internucleoside linkage, or modified nucleobase. A “modified nucleoside” means a nucleoside having, independently, a modified sugar moiety or modified nucleobase.

“Modified oligonucleotide” means an oligonucleotide comprising at least one modified nucleotide.

“Modified sugar” refers to a substitution or change from a natural sugar.

“Motif” means the pattern of chemically distinct regions in an antisense compound.

“Naturally occurring internucleoside linkage” means a 3′ to 5′ phosphodiester linkage.

“Natural sugar moiety” means a sugar found in DNA (2′-H) or RNA (2′-OH).

“Nucleic acid” refers to molecules composed of monomeric nucleotides. A nucleic acid includes ribonucleic acids (RNA), deoxyribonucleic acids (DNA), single-stranded nucleic acids, double-stranded nucleic acids, small interfering ribonucleic acids (siRNA), and microRNAs (miRNA). A nucleic acid can also comprise a combination of these elements in a single molecule.

“Nucleobase” means a heterocyclic moiety capable of pairing with a base of another nucleic acid.

“Nucleobase sequence” means the order of contiguous nucleobases independent of any sugar, linkage, or nucleobase modification.

“Nucleoside” means a nucleobase linked to a sugar.

“Nucleotide” means a nucleoside having a phosphate group covalently linked to the sugar portion of the nucleoside.

“Oligomeric compound” or “oligomer” means a polymer of linked monomeric subunits which is capable of hybridizing to at least a region of a nucleic acid molecule.

“Oligonucleotide” means a polymer of linked nucleosides each of which can be modified or unmodified, independent one from another.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration. Administration can be continuous, or chronic, or short or intermittent.

“PCSK9” (also known as “proprotein convertase subtilisin/kexin type 9”) is an enzyme which plays a major regulatory role in cholesterol homeostasis. PCSK9 binds to low-density lipoprotein receptors (LDLR), inducing LDLR degradation. Reduced LDLR levels result in decreased metabolism of low-density lipoproteins, which could lead to cardiovascular disease.

“Peptide” means a molecule formed by linking at least two amino acids by amide bonds. Peptide refers to polypeptides and proteins.

“Pharmaceutical composition” means a mixture of substances suitable for administering to an individual. For example, a pharmaceutical composition can comprise one or more active agents and a sterile aqueous solution.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e. linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevent” refers to delaying or forestalling the onset or development of a disease, disorder, or condition for a period of time from minutes to indefinitely. Prevent also means reducing risk of developing a disease, disorder, or condition.

“Prodrug” means a therapeutic agent that is prepared in an inactive form that is converted to an active form within the body or cells thereof by the action of endogenous enzymes or other chemicals or conditions.

“Side effects” means physiological responses attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum can indicate liver toxicity or liver function abnormality. For example, increased bilirubin can indicate liver toxicity or liver function abnormality.

“Single-stranded oligonucleotide” means an oligonucleotide which is not hybridized to a complementary strand.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e. under physiological conditions in the case of in vivo assays and therapeutic treatments.

“Targeting” or “targeted” means the process of design and selection of an antisense compound that will specifically hybridize to a target nucleic acid and induce a desired effect.

“Target nucleic acid,” “target RNA,” and “target RNA transcript” all refer to a nucleic acid capable of being targeted by antisense compounds.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Therapeutically effective amount” means an amount of an agent that provides a therapeutic benefit to an individual.

“Treat” refers to administering a pharmaceutical composition to effect an alteration or improvement of a disease, disorder, or condition.

“TTC39” means any of the isoforms TTC39A, TTC39B or TTC39C. TTC39 can mean a nucleic acid or protein of any of the isoforms.

“TTC39 expression” means the level of mRNA transcribed from the gene encoding TTC39 or the level of protein translated from the mRNA. TTC39 expression can be determined by art known methods such as a Northern or Western blot.

“TTC39 nucleic acid” means any nucleic acid encoding a tetratricopeptide repeat domain 39 (“TTC39”) isoform. For example, in certain embodiments, a TTC39 nucleic acid includes a DNA sequence encoding TTC39, an RNA sequence transcribed from DNA encoding TTC39 (including genomic DNA comprising introns and exons), and an mRNA sequence encoding TTC39. “TTC39 mRNA” means an mRNA encoding a TTC39 protein. “TTC39A mRNA” means an mRNA encoding a TTC39A protein. “TTC39B mRNA” means an mRNA encoding a TTC39B protein. “TTC39C mRNA” means an mRNA encoding a TTC39C protein.

“Unmodified nucleotide” means a nucleotide composed of naturally occurring nucleobases, sugar moieties, and internucleoside linkages. In certain embodiments, an unmodified nucleotide is an RNA nucleotide (i.e. β-D-ribonucleosides) or a DNA nucleotide (i.e. 13-D-deoxyribonucleoside).

### Certain Embodiments

Certain embodiments provide methods, compounds, and compositions for inhibiting TTC39 expression.

Certain embodiments provide a method of reducing a TTC39 isoform expression in an animal comprising administering to the animal a compound comprising a modified oligonucleotide targeted to the TTC39 isoform. In certain embodiments, the modified oligonucleotide is 12 to 30 linked nucleosides in length.

Certain embodiments provide a method of increasing HDL level in an animal comprising administering to the animal a compound comprising a modified oligonucleotide targeted to a TTC39 isoform, wherein the modified oligonucleotide reduces the TTC39 isoform expression in the animal, thereby increasing the HDL level in the animal. In certain embodiments, the modified oligonucleotide is 12 to 30 linked nucleosides in length

Certain embodiments provide a method of decreasing PCSK9 level in an animal comprising administering to the animal a compound comprising a modified oligonucleotide targeted to a TTC39 isoform, wherein the modified oligonucleotide reduces the TTC39 isoform expression in the animal, thereby reducing the PCSK9 level in the animal. In certain embodiments, the modified oligonucleotide is 12 to 30 linked nucleosides in length.

Certain embodiments provide a method of increasing apoA1 level in an animal comprising administering to the animal a compound comprising a modified oligonucleotide targeted to a TTC39 isoform, wherein the modified oligonucleotide reduces the TTC39 isoform expression in the animal, thereby increasing the apoA1 level in the animal. In certain embodiments, the modified oligonucleotide is 12 to 30 linked nucleosides in length.

Certain embodiments provide a method of increasing apoA4 level in an animal comprising administering to the animal a compound comprising a modified oligonucleotide targeted to a TTC39 isoform, wherein the modified oligonucleotide reduces the TTC39 isoform expression in the animal, thereby increasing the apoA4 level in the animal. In certain embodiments, the modified oligonucleotide is 12 to 30 linked nucleosides in length.

Certain embodiments provide a method of increasing LDL receptor level in an animal comprising administering to the animal a compound comprising a modified oligonucleotide targeted to a TTC39 isoform, wherein the modified oligonucleotide reduces the TTC39 isoform expression in the animal, thereby increasing the LDL receptor level in the animal. In certain embodiments, the modified oligonucleotide is 12 to 30 linked nucleosides in length.

Certain embodiments provide a method for treating an animal with cardiovascular disease comprising: a) identifying said animal with cardiovascular disease, and b) administering to said animal a therapeutically effective amount of a compound comprising a modified oligonucleotide targeted to a TTC39 isoform. In certain embodiments, the therapeutically effective amount of the compound administered to the animal increases HDL in the animal. In certain embodiments, the modified oligonucleotide is 12 to 30 linked nucleosides in length.

In certain embodiments, the TTC39 isoform is isoform A, B or C. In certain embodiments, the TTC39A isoform has the sequence as set forth in GenBank Accession No. NM_{—}001144832.1 (incorporated herein as SEQ ID NO: 2). In certain embodiments, the TTC39β isoform has the sequence as set forth in GenBank Accession No. NM_{—}152574.1 (incorporated herein as SEQ ID NO: 1). In certain embodiments, the TTC39C isoform has the sequence as set forth in GenBank Accession No. NM_{—}153211.3 (incorporated herein as SEQ ID NO: 3).

Certain embodiments provide a method of reducing TTC39B expression comprising administering to an animal a compound comprising a modified oligonucleotide consisting of 12 to 30 linked nucleosides and having a nucleobase sequence at least 90% complementary to SEQ ID NO: 1 as measured over the entirety of said modified oligonucleotide. In certain embodiments, the reduction in TTC39B expression increases HDL, apoA1, apoA4 and/or LDL receptor levels in the animal. In certain embodiments, the reduction in TTC39B expression decreases PCSK9 levels in the animal.

Certain embodiments provide a method of increasing HDL in an animal comprising administering to the animal a compound comprising a modified oligonucleotide which reduces expression of TTC39B, wherein the modified oligonucleotide consists of 12 to 30 linked nucleosides having a nucleobase sequence at least 90% complementary to SEQ ID NO: 1 as measured over the entirety of said modified oligonucleotide, and wherein said reduction of TTC39B increases HDL in the animal.

Certain embodiments provide a method of decreasing PCSK9 in an animal comprising administering to the animal a compound comprising a modified oligonucleotide which reduces expression of TTC39B, wherein the modified oligonucleotide consists of 12 to 30 linked nucleosides having a nucleobase sequence at least 90% complementary to SEQ ID NO: 1 as measured over the entirety of said modified oligonucleotide, and wherein said reduction of TTC39B decreases PCSK9 in the animal.

Certain embodiments provide a method of increasing apoA1 in an animal comprising administering to the animal a compound comprising a modified oligonucleotide which reduces expression of TTC39B, wherein the modified oligonucleotide consists of 12 to 30 linked nucleosides having a nucleobase sequence at least 90% complementary to SEQ ID NO: 1 as measured over the entirety of said modified oligonucleotide, and wherein said reduction of TTC39B increases apoA1 in the animal.

Certain embodiments provide a method of increasing apoA4 in an animal comprising administering to the animal a compound comprising a modified oligonucleotide which reduces expression of TTC39B, wherein the modified oligonucleotide consists of 12 to 30 linked nucleosides having a nucleobase sequence at least 90% complementary to SEQ ID NO: 1 as measured over the entirety of said modified oligonucleotide, and wherein said reduction of TTC39B increases apoA4 in the animal.

Certain embodiments provide a method of increasing LDL receptor in an animal comprising administering to the animal a compound comprising a modified oligonucleotide which reduces expression of TTC39B, wherein the modified oligonucleotide consists of 12 to 30 linked nucleosides having a nucleobase sequence at least 90% complementary to SEQ ID NO: 1 as measured over the entirety of said modified oligonucleotide, and wherein said reduction of TTC39B increases LDL receptor in the animal.

Certain embodiments provide a method for treating an animal with cardiovascular disease comprising: a) identifying said animal with cardiovascular disease, and b) administering to said animal a therapeutically effective amount of a compound comprising a modified oligonucleotide consisting of 12 to 30 linked nucleosides and having a nucleobase sequence at least 90% complementary to SEQ ID NO: 1 as measured over the entirety of said modified oligonucleotide. In certain embodiments, the therapeutically effective amount of the compound administered to the animal increases HDL, apoA1, apoA4 and/or LDL receptor in the animal. In certain embodiments, the therapeutically effective amount of the compound administered to the animal decreases PCSK9 in the animal.

In certain embodiments, the modified oligonucleotide has a nucleobase sequence comprising at least 8 contiguous nucleobases of the nucleobase sequence recited in SEQ ID NO: 1, 2 or 3.

In certain embodiments, the animal is a human.

In certain embodiments, the compounds or compositions of the invention are designated as a first agent and the methods of the invention further comprise administering a second agent. In certain embodiments, the first agent and the second agent are co-administered. In certain embodiments the first agent and the second agent are co-administered sequentially or concomitantly.

In certain embodiments, administration comprises parenteral administration.

In certain embodiments, the compound of the invention consists of a single-stranded modified oligonucleotide. In certain embodiments, the nucleobase sequence of the modified oligonucleotide is at least 95% complementary to SEQ ID NO: 1, 2 or 3 as measured over the entirety of said modified oligonucleotide. In certain embodiments, the nucleobase sequence of the modified oligonucleotide is at least 100% complementary to SEQ ID NO: 1, 2 or 3 as measured over the entirety of said modified oligonucleotide.

In certain embodiments, at least one internucleoside linkage of said modified oligonucleotide is a modified internucleoside linkage. In certain embodiments, each internucleoside linkage is a phosphorothioate internucleoside linkage.

In certain embodiments, at least one nucleoside of said modified oligonucleotide comprises a modified sugar. In certain embodiments, at least one modified sugar is a bicyclic sugar. In certain embodiments, at least one modified sugar comprises a 2′-O-methoxyethyl or a 4′-(CH₂)ₙ—O-2′ bridge, wherein n is 1 or 2.

In certain embodiments, at least one nucleoside of said modified oligonucleotide comprises a modified nucleobase. In certain embodiments, the modified nucleobase is a 5-methylcytosine.

In certain embodiments, the modified oligonucleotide comprises: a) a gap segment consisting of linked deoxynucleosides; b) a 5′ wing segment consisting of linked nucleosides; and c) a 3′ wing segment consisting of linked nucleosides. The gap segment is positioned between the 5′ wing segment and the 3′ wing segment and each nucleoside of each wing segment comprises a modified sugar.

In certain embodiments, the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, and each cytosine in said modified oligonucleotide is a 5′-methylcytosine.

In certain embodiments, the modified oligonucleotide consists of 20 linked nucleosides.

Certain embodiments provide a method of reducing TTC39A in an animal comprising administering to the animal a modified oligonucleotide which reduces expression of TTC39A, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine.

Certain embodiments provide a method of increasing HDL in an animal comprising administering to the animal a compound comprising a modified oligonucleotide which reduces expression of TTC39A, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine and said reduction of TTC39A expression increases HDL in the animal.

Certain embodiments provide a method of reducing PCSK9 in an animal comprising administering to the animal a compound comprising a modified oligonucleotide which reduces expression of TTC39A, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine and reduction of TTC39A expression reduces PCSK9 in the animal.

Certain embodiments provide a method of increasing apoA1 in an animal comprising administering to the animal a compound comprising a modified oligonucleotide which reduces expression of TTC39A, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine and said reduction of TTC39A expression increases apoA1 in the animal.

Certain embodiments provide a method of increasing apoA4 in an animal comprising administering to the animal a compound comprising a modified oligonucleotide which reduces expression of TTC39A, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine and said reduction of TTC39A expression increases apoA4 in the animal.

Certain embodiments provide a method of increasing LDL receptor in an animal comprising administering to the animal a compound comprising a modified oligonucleotide which reduces expression of TTC39A, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine and said reduction of TTC39A expression increases LDL receptor in the animal.

Certain embodiments provide a method of treating cardiovascular disease in an animal comprising administering to the animal a compound comprising a modified oligonucleotide which reduces expression of TTC39A, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine and reduction of TTC39A expression treats cardiovascular disease in the animal.

Certain embodiments provide a method of reducing TTC39B in an animal comprising administering to the animal a modified oligonucleotide which reduces expression of TTC39B, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine.

Certain embodiments provide a method of increasing HDL in an animal comprising administering to the animal a compound comprising a modified oligonucleotide which reduces expression of TTC39B, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine and said reduction of TTC39B expression increases HDL in the animal.

Certain embodiments provide a method of reducing PCSK9 in an animal comprising administering to the animal a compound comprising a modified oligonucleotide which reduces expression of TTC39B, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine and reduction of TTC39B expression reduces PCSK9 in the animal.

Certain embodiments provide a method of increasing apoA1 in an animal comprising administering to the animal a compound comprising a modified oligonucleotide which reduces expression of TTC39B, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine and said reduction of TTC39B expression increases apoA1 in the animal.

Certain embodiments provide a method of increasing apoA4 in an animal comprising administering to the animal a compound comprising a modified oligonucleotide which reduces expression of TTC39B, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine and said reduction of TTC39B expression increases apoA4 in the animal.

Certain embodiments provide a method of increasing LDL receptor in an animal comprising administering to the animal a compound comprising a modified oligonucleotide which reduces expression of TTC39B, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine and said reduction of TTC39B expression increases LDL receptor in the animal.

Certain embodiments provide a method of treating cardiovascular disease in an animal comprising administering to the animal a compound comprising a modified oligonucleotide which reduces expression of TTC39B, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine and reduction of TTC39B expression treats cardiovascular disease in the animal.

Certain embodiments provide a method of reducing TTC39C in an animal comprising administering to the animal a modified oligonucleotide which reduces expression of TTC39C, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine.

Certain embodiments provide a method of increasing HDL in an animal comprising administering to the animal a compound comprising a modified oligonucleotide which reduces expression of TTC39C, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine and said reduction of TTC39C expression increases HDL in the animal.

Certain embodiments provide a method of reducing PCSK9 in an animal comprising administering to the animal a compound comprising a modified oligonucleotide which reduces expression of TTC39C, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine and reduction of TTC39C expression reduces PCSK9 in the animal.

Certain embodiments provide a method of increasing apoA1 in an animal comprising administering to the animal a compound comprising a modified oligonucleotide which reduces expression of TTC39C, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine and said reduction of TTC39C expression increases apoA1 in the animal.

Certain embodiments provide a method of increasing apoA4 in an animal comprising administering to the animal a compound comprising a modified oligonucleotide which reduces expression of TTC39C, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine and said reduction of TTC39C expression increases apoA4 in the animal.

Certain embodiments provide a method of increasing LDL receptor in an animal comprising administering to the animal a compound comprising a modified oligonucleotide which reduces expression of TTC39C, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine and said reduction of TTC39C expression increases LDL receptor in the animal.

Certain embodiments provide a method of treating cardiovascular disease in an animal comprising administering to the animal a compound comprising a modified oligonucleotide which reduces expression of TTC39C, wherein the modified oligonucleotide comprises: a) a gap segment consisting of ten linked deoxynucleosides; b) a 5′ wing segment consisting of five linked nucleosides; and c) a 3′ wing segment consisting of five linked nucleosides. The gap segment is positioned between the 5′ wing segment and the 3′ wing segment, each nucleoside of each wing segment comprises a 2′-O-methoxyethyl sugar, each internucleoside linkage of said modified oligonucleotide is a phosphorothioate linkage, each cytosine in said modified oligonucleotide is a 5′-methylcytosine and reduction of TTC39C expression treats cardiovascular disease in the animal.

In certain embodiments, cardiovascular disease can be, but is not limited to, arteriosclerosis, atherosclerosis, coronary heart disease, heart failure, hypertension, dyslipidemia, hypercholesterolemia, acute coronary syndrome, type II diabetes, type II diabetes with dyslipidemia, hepatic steatosis, non-alcoholic steatohepatitis, non-alcoholic fatty liver disease, hypertriglyceridemia, hyperfattyacidemia, hyperlipidemia and metabolic syndrome.

Certain embodiments of the invention provide the use of a compound targeted to TTC39A as described herein in the manufacture of a medicament for reducing TTC39A, increasing HDL, reducing PCSK9, increasing apoA1, increasing apoA4, increasing LDL receptor and/or for treating, ameliorating, or preventing cardiovascular disease.

Certain embodiments of the invention provide the use of a compound targeted to TTC39B as described herein in the manufacture of a medicament for reducing TTC39B, increasing HDL, reducing PCSK9, increasing apoA1, increasing apoA4, increasing LDL receptor and/or for treating, ameliorating, or preventing cardiovascular disease.

Certain embodiments of the invention provide the use of a compound targeted to TTC39C as described herein in the manufacture of a medicament for reducing TTC39C, increasing HDL, reducing PCSK9, increasing apoA1, increasing apoA4, increasing LDL receptor and/or for treating, ameliorating, or preventing cardiovascular disease.

Certain embodiments of the invention provide a kit for reducing TTC39A, increasing HDL, reducing PCSK9, increasing apoA1, increasing apoA4, increasing LDL receptor and/or for treating, preventing, or ameliorating cardiovascular disease as described herein wherein the kit comprises: a) a compound targeting TTC39A as described herein; and optionally b) an additional agent or therapy as described herein. The kit can further include instructions or a label for using the kit to treat, prevent, or ameliorate cardiovascular disease.

Certain embodiments of the invention provide a kit for reducing TTC39B, increasing HDL, reducing PCSK9, increasing apoA1, increasing apoA4, increasing LDL receptor and/or for treating, preventing, or ameliorating cardiovascular disease as described herein wherein the kit comprises: a) a compound targeting TTC39B as described herein; and optionally b) an additional agent or therapy as described herein. The kit can further include instructions or a label for using the kit to treat, prevent, or ameliorate cardiovascular disease.

Certain embodiments of the invention provide a kit for reducing TTC39C, increasing HDL, reducing PCSK9, increasing apoA1, increasing apoA4, increasing LDL receptor and/or for treating, preventing, or ameliorating cardiovascular disease as described herein wherein the kit comprises: a) a compound targeting TTC39C as described herein; and optionally b) an additional agent or therapy as described herein. The kit can further include instructions or a label for using the kit to treat, prevent, or ameliorate cardiovascular disease.

### Antisense Compounds

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound can be “antisense” to a target nucleic acid, meaning that is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to a TTC39 nucleic acid is 12 to 30 nucleotides in length. In other words, antisense compounds are from 12 to 30 linked nucleobases. In other embodiments, the antisense compound comprises a modified oligonucleotide consisting of 8 to 80, 12 to 50, 15 to 30, 18 to 24, 19 to 22, or 20 linked nucleobases. In certain such embodiments, the antisense compound comprises a modified oligonucleotide consisting of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked nucleobases in length, or a range defined by any two of the above values.

In certain embodiments, the antisense compound comprises a shortened or truncated modified oligonucleotide. The shortened or truncated modified oligonucleotide can have a single nucleoside deleted from the 5′ end (5′ truncation), or alternatively from the 3′ end (3′ truncation). A shortened or truncated oligonucleotide can have two nucleosides deleted from the 5′ end, or alternatively can have two subunits deleted from the 3′ end. Alternatively, the deleted nucleosides can be dispersed throughout the modified oligonucleotide, for example, in an antisense compound having one nucleoside deleted from the 5′ end and one nucleoside deleted from the 3′ end.

When a single additional nucleoside is present in a lengthened oligonucleotide, the additional nucleoside can be located at the 5′ or 3′ end of the oligonucleotide. When two or more additional nucleosides are present, the added nucleosides can be adjacent to each other, for example, in an oligonucleotide having two nucleosides added to the 5′ end (5′ addition), or alternatively to the 3′ end (3′ addition), of the oligonucleotide. Alternatively, the added nucleoside can be dispersed throughout the antisense compound, for example, in an oligonucleotide having one nucleoside added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Antisense Compound Motifs

In certain embodiments, antisense compounds targeted to a TTC39 nucleic acid have chemically modified subunits arranged in patterns, or motifs, to confer to the antisense compounds properties such as enhanced the inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound can optionally serve as a substrate for the cellular endonuclease RNase H, which cleaves the RNA strand of an RNA:DNA duplex.

Antisense compounds having a gapmer motif are considered chimeric antisense compounds. In a gapmer an internal region having a plurality of nucleotides that supports RNase H cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer can in some embodiments include β-D-ribonucleosides, β-D-deoxyribonucleosides, 2′-modified nucleosides (such 2′-modified nucleosides can include 2′-MOE, and 2′-O—CH₃, among others), and bicyclic sugar modified nucleosides (such bicyclic sugar modified nucleosides can include those having a 4′-(CH2)n-O-2′ bridge, where n=1 or n=2). Preferably, each distinct region comprises uniform sugar moieties. The wing-gap-wing motif is frequently described as “X-Y-Z”, where “X” represents the length of the 5′ wing region, “Y” represents the length of the gap region, and “Z” represents the length of the 3′ wing region. As used herein, a gapmer described as “X-Y-Z” has a configuration such that the gap segment is positioned immediately adjacent each of the 5′ wing segment and the 3′ wing segment. Thus, no intervening nucleotides exist between the 5′ wing segment and gap segment, or the gap segment and the 3′ wing segment. Any of the antisense compounds described herein can have a gapmer motif. In some embodiments, X and Z are the same, in other embodiments they are different. In a preferred embodiment, Y is between 8 and 15 nucleotides. X, Y or Z can be any of 1, 2, 3, 4, 5, 6, 7, 8, 9, 10, 11, 12, 13, 14, 15, 16, 17, 18, 19, 20, 25, 30 or more nucleotides. Thus, gapmers include, but are not limited to, for example 5-10-5, 4-8-4, 4-12-3, 4-12-4, 3-14-3, 2-13-5, 2-16-2, 1-18-1, 3-10-3, 2-10-2, 1-10-1, 2-8-2, 6-8-6 or 5-8-5.

In certain embodiments, the antisense compound as a “wingmer” motif, having a wing-gap or gap-wing configuration, i.e. an X-Y or Y-Z configuration as described above for the gapmer configuration. Thus, wingmer configurations include, but are not limited to, for example 5-10, 8-4, 4-12, 12-4, 3-14, 16-2, 18-1, 10-3, 2-10, 1-10, 8-2, 2-13, or 5-13.

In certain embodiments, antisense compounds targeted to a TTC39 nucleic acid possess a 5-10-5 gapmer motif.

In certain embodiments, an antisense compound targeted to a TTC39 nucleic acid has a gap-widened motif.

In certain embodiments, an antisense oligonucleotide targeted to a TTC39 nucleic acid has a gap segment of ten 2′-deoxyribonucleotides positioned immediately adjacent to and between wing segments of five chemically modified nucleosides. In certain embodiments, the chemical modification comprises a 2′-sugar modification. In another embodiment, the chemical modification comprises a 2′-MOE sugar modification.

### Target Nucleic Acids, Target Regions and Nucleotide Sequences

Nucleotide sequences that encode TTC39 include, without limitation, the following. The TTC39A isoform can have the human sequence as set forth in GenBank Accession No. NM_{—}001144832.1 (incorporated herein as SEQ ID NO: 2) or the murine sequences as set forth in GenBank Accession No. NM_{—}001145948.1 (incorporated herein as SEQ ID NO: 4) and GenBank Accession No. NM_{—}001134519.1 (incorporated herein as SEQ ID NO: 5). The TTC39β isoform can have the human sequence as set forth in GenBank Accession No. NM_{—}152574.1 (incorporated herein as SEQ ID NO: 1) and GenBank Accession No. NT_{—}008413.17, position 15158001 to 15300000 (SEQ ID NO: 15) or the murine sequences as set forth in GenBank Accession No. NM_{—}027238.2 (incorporated herein as SEQ ID NO: 6) and GenBank Accession No. NM_{—}001106665.1 (incorporated herein as SEQ ID NO: 7). The TTC39C isoform has the human sequence as set forth in GenBank Accession No. NM_{—}153211.3 (incorporated herein as SEQ ID NO: 3) or NM_{—}028341.4 (incorporated herein as SEQ ID NO: 8) and GenBank Accession No. NM_{—}001077231.1 (incorporated herein as SEQ ID NO: 9).

It is understood that the sequence set forth in each SEQ ID NO in the Examples contained herein is independent of any modification to a sugar moiety, an internucleoside linkage, or a nucleobase. As such, antisense compounds defined by a SEQ ID NO can comprise, independently, one or more modifications to al sugar moiety, an internucleoside linkage, or a nucleobase. Antisense compounds described by Isis Number (Isis No) indicate a combination of nucleobase sequence and motif.

In certain embodiments, a target region is a structurally defined region of the target nucleic acid. For example, a target region can encompass a 3′ UTR, a 5′ UTR, an exon, an intron, an exon/intron junction, a coding region, a translation initiation region, translation termination region, or other defined nucleic acid region. The structurally defined regions for TTC39 can be obtained by accession number from sequence databases such as NCBI and such information is incorporated herein by reference. In certain embodiments, a target region can encompass the sequence from a 5′ target site of one target segment within the target region to a 3′ target site of another target segment within the target region.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region can contain one or more target segments. Multiple target segments within a target region can be overlapping. Alternatively, they can be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein. The target region starting at any of the 5′ or 3′ targets sites listed herein can extend 250, 200, 150, 100, 90, 80, 70, 60, 50, 40, 30, 20, or 10 nucleotides upstream or downstream of the site.

Suitable target segments can be found within a 5′ UTR, a coding region, a 3′ UTR, an intron, an exon, or an exon/intron junction. Target segments containing a start codon or a stop codon are also suitable target segments. A suitable target segment can specifically exclude a certain structurally defined region such as the start codon or stop codon.

The determination of suitable target segments can include a comparison of the sequence of a target nucleic acid to other sequences throughout the genome. For example, the BLAST algorithm can be used to identify regions of similarity amongst different nucleic acids. This comparison can prevent the selection of antisense compound sequences that can hybridize in a non-specific manner to sequences other than a selected target nucleic acid (i.e., non-target or off-target sequences).

There can be variation in activity (e.g., as defined by percent reduction of target nucleic acid levels) of the antisense compounds within an active target region. In certain embodiments, reductions in TTC39 mRNA levels are indicative of inhibition of TTC39 protein expression. Reductions in levels of a TTC39 protein are also indicative of inhibition of target mRNA expression. Further, phenotypic changes, such as an increase in HDL, apoA1 or apoA4 levels, or a decrease in PCSK9 levels can be indicative of inhibition of TTC39 mRNA and/or protein expression.

### Hybridization

In some embodiments, hybridization occurs between an antisense compound disclosed herein and a TTC39 nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art (Sambrooke and Russell, Molecular Cloning: A Laboratory Manual, 3^{rd} Ed., 2001). In certain embodiments, the antisense compounds provided herein are specifically hybridizable with a TTC39 nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a TTC39 nucleic acid).

An antisense compound can hybridize over one or more segments of a TTC39 nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a TTC39 nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining noncomplementary nucleobases can be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e. 100% complementary) to a target nucleic acid, or specified portion thereof. For example, antisense compound can be fully complementary to a TTC39 nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound can be fully complementary to the target sequence, depending on whether the remaining 10 nucleobases of the antisense compound are also complementary to the target sequence.

The location of a non-complementary nucleobase can be at the 5′ end or 3′ end of the antisense compound. Alternatively, the non-complementary nucleobase or nucleobases can be at an internal position of the antisense compound. When two or more non-complementary nucleobases are present, they can be either contiguous (i.e. linked) or non-contiguous. In one embodiment, a non-complementary nucleobase is located in the wing segment of a gapmer antisense oligonucleotide.

In certain embodiments, antisense compounds that are, or are up to, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a TTC39 nucleic acid, or specified portion thereof.

In certain embodiments, antisense compounds that are, or are up to, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a TTC39 nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds are complementary to at least an 8 nucleobase portion of a target segment. In certain embodiments, the antisense compounds are complementary to at least a 12 nucleobase portion of a target segment. In certain embodiments, the antisense compounds are complementary to at least a 15 nucleobase portion of a target segment. Also contemplated are antisense compounds that are complementary to at least an 8, 9, 10, 11, 12, 13, 14, 15, 16, 17, 18, 19, 20, or more nucleobase portion of a target segment, or a range defined by any two of these values.

### Identity

The antisense compounds provided herein can also have a defined percent identity to a particular nucleotide sequence, SEQ ID NO, or compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases can be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides can also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a TTC39 nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

### Modified Sugar Moieties

Antisense compounds of the invention can optionally contain one or more nucleosides wherein the sugar group has been modified. Such sugar modified nucleosides may impart enhanced nuclease stability, increased binding affinity or some other beneficial biological property to the antisense compounds. In certain embodiments, nucleosides comprise a chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R1)(R)2 (R═H, C1-C12 alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F, 2′-OCH3 and 2′-O(CH2)2OCH3 substituent groups. The substituent at the 2′ position can also be selected from allyl, amino, azido, thio, O-allyl, O—C1-C10 alkyl, OCF3, O(CH2)2SCH3, O(CH2)2-O—N(Rm)(Rn), and O—CH2-C(═O)—N(Rm)(Rn), where each Rm and Rn is, independently, H or substituted or unsubstituted C1-C10 alkyl, O-alkaryl or O-aralkyl, substituted alkyl, alkenyl, alkynyl, alkaryl, aralkyl, SH, SCH₃, OCN, Cl, Br, CN, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an antisense compound, and other substituents having similar properties

Examples of bicyclic nucleic acids (BNAs) include without limitation nucleosides comprising a bridge between the 4′ and the 2′ ribosyl ring atoms. In certain embodiments, antisense compounds provided herein include one or more BNA nucleosides wherein the bridge comprises one of the formulas: 4′-(CH2)-O-2′ (LNA); 4′-(CH2)-S-2; 4′-(CH2)2-O-2′ (ENA); 4′-C(CH3)2-O-2′ (see PCT/US2008/068922); 4′-CH(CH3)-O-2′ and 4′-CH(CH₂OCH3)-O-2′ (see U.S. Pat. No. 7,399,845, issued on Jul. 15, 2008); 4′-CH2-N(OCH3)-2′ (see PCT/US2008/064591); 4′-CH2-O—N(CH3)-2′ (see published U.S. Patent Application US2004-0171570, published Sep. 2, 2004); 4′-CH2-N(R)—O-2′ (see U.S. Pat. No. 7,427,672, issued on Sep. 23, 2008); 4′-CH2-CH(CH3)-2′ (see Chattopadhyaya et al., J. Org. Chem., 2009, 74, 118-134) and 4′-CH2-C(═CH2)-2′ (see PCT/US2008/066154); and wherein R is, independently, H, C1-C12 alkyl, or a protecting group. Each of the foregoing BNAs include various stereochemical sugar configurations including for example α-L-ribofuranose and β-D-ribofuranose (see PCT international application PCT/DK98/00393, published on Mar. 25, 1999 as WO 99/14226). Previously, α-L-methyleneoxy (4′-CH₂—O-2′) BNA's have also been incorporated into antisense oligonucleotides that showed antisense activity (Frieden et al., Nucleic Acids Research, 2003, 21, 6365-6372).

Further reports related to bicyclic nucleosides can be found in published literature (see for example: Srivastava et al., J. Am. Chem. Soc., 2007, 129, 8362-8379; U.S. Pat. Nos. 7,053,207; 6,268,490; 6,770,748; 6,794,499; 7,034,133; and 6,525,191; Elayadi et al., Curr. Opinion Invens. Drugs, 2001, 2, 558-561; Braasch et al., Chem. Biol., 2001, 8, 1-7; and Orum et al., Curr. Opinion Mol. Ther., 2001, 3, 239-243; and U.S. Pat. No. 6,670,461; International applications WO 2004/106356; WO 94/14226; WO 2005/021570; U.S. Patent Publication Nos. US2004-0171570; US2007-0287831; US2008-0039618; U.S. Pat. No. 7,399,845; U.S. patent Ser. Nos. 12/129,154; 60/989,574; 61/026,995; 61/026,998; 61/056,564; 61/086,231; 61/097,787; 61/099,844; PCT International Applications Nos. PCT/US2008/064591; PCT/US2008/066154; PCT/US2008/068922; and Published PCT International Applications WO 2007/134181).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═O)—, —C(═NRₐ)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is, —[C(Rₐ)(R_{b})]ₙ—, —[C(Rₐ)(R_{b})]ₙ—O—, —C(RₐR_{b})—N(R)—O— or —C(RₐR_{b})—O—N(R)—. In certain embodiments, the bridge is 4′-CH₂-2′, 4′-(CH₂)₃-2′, 4′-CH₂—O-2′, 4′-(CH₂)₂—O-2′, 4′-CH₂—O—N(R)-2′ and 4′-CH₂—N(R)—O-2′-wherein each R is, independently, H, a protecting group or C₁-C₁₂ alkyl.

In certain embodiments, bicyclic nucleosides include, but are not limited to, (A) α-L-Methyleneoxy (4′-CH₂—O-2′) BNA, (B) β-D-Methyleneoxy (4′-CH₂—O-2′) BNA, (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, and (F) Methyl(methyleneoxy) (4′-CH(CH₃)—O-2′) BNA, (G) Methylene-thio (4′-CH₂—S-2′) BNA, (H) Methylene-amino (4′-CH₂—N(R)-2′) BNA, (I) Methyl carbocyclic (4′-CH₂—CH(CH₃)-2′) BNA, and (J) Propylene carbocyclic (4′-(CH₂)₃-2′) BNA as depicted below.

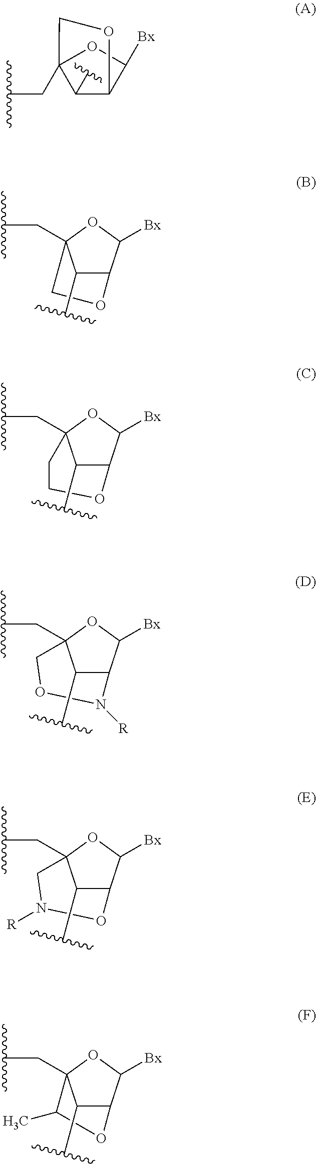

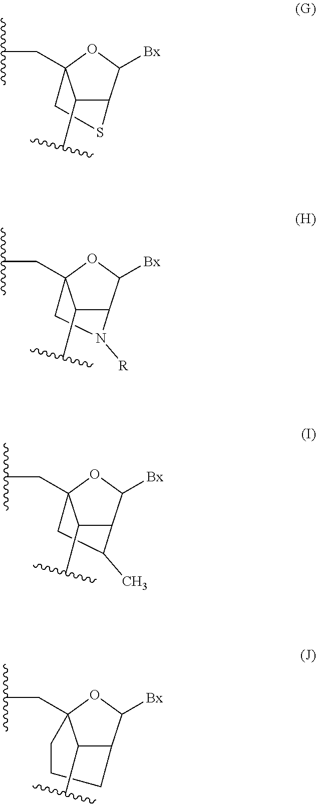

wherein Bx is the base moiety and R is independently H, a protecting group or C₁-C₁₂ alkyl.

In certain embodiments, bicyclic nucleoside having Formula I:

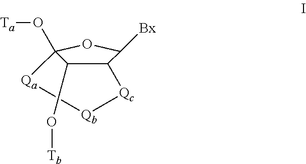

wherein:

Bx is a heterocyclic base moiety;

-Qₐ-Q_{b}-Q_{c}- is —CH₂—N(R_{c})—CH₂—, —C(═O)—N(R_{c})—CH₂—, —CH₂—O—N(R_{c})—, —CH₂—N(R_{c})—O— or —N(R_{c})—O—CH₂;

R_{c} is C₁-C₁₂ alkyl or an amino protecting group; and

Tₐ and T_{b} are each, independently H, a hydroxyl protecting group, a conjugate group, a reactive phosphorus group, a phosphorus moiety or a covalent attachment to a support medium.

In certain embodiments, bicyclic nucleoside having Formula II:

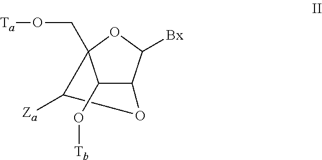

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol or substituted thio.

In one embodiment, each of the substituted groups is, independently, mono or poly substituted with substituent groups independently selected from halogen, oxo, hydroxyl, OJ_{c}, NJ_{c}J_{d}, SJ_{c}, N₃, OC(═X)J_{c}, and NJ_{c}C(═X)NJ_{c}J_{d}, wherein each J_{c}, J_{d} and Jₑ is, independently, H, C₁-C₆ alkyl, or substituted C₁-C₆ alkyl and X is O or NJ_{c}.

In certain embodiments, bicyclic nucleoside having Formula III:

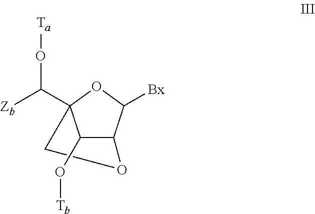

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(═O)—).

In certain embodiments, bicyclic nucleoside having Formula IV:

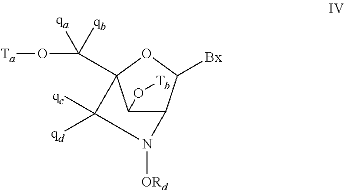

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

R_{d} is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;

each qₐ, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

In certain embodiments, bicyclic nucleoside having Formula V:

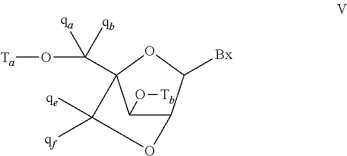

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

qₐ, q_{b}, qₑ and g_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;

or qₑ and g_{f} together are ═C(q_{g})(qₕ);

q_{g} and qₕ are each, independently, H, halogen, C₁-C₁₂ alkyl or substituted C₁-C₁₂ alkyl.

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of T-amino-BNA, a novel comformationally restricted high-affinity oligonucleotide analog has been described in the art (Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported.

In certain embodiments, bicyclic nucleoside having Formula VI:

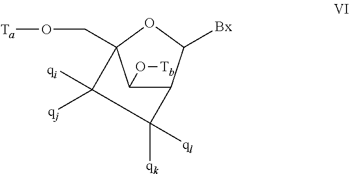

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

each qᵢ, qⱼ, qₖ and qₗ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ; and

qᵢ and qⱼ or qₗ and qₖ together are ═C(q_{g})(qₕ), wherein q_{g} and qₕ are each, independently, H, halogen, C₁-C₁₂ alkyl or substituted C₁-C₁₂ alkyl.

One carbocyclic bicyclic nucleoside having a 4′-(CH₂)₃-2′ bridge and the alkenyl analog bridge 4′-CH═CH—CH₂-2′ have been described (Freier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc., 2007, 129(26), 8362-8379).

In certain embodiments, nucleosides are modified by replacement of the ribosyl ring with a sugar surrogate. Such modification includes without limitation, replacement of the ribosyl ring with a surrogate ring system (sometimes referred to as DNA analogs) such as a morpholino ring, a cyclohexenyl ring, a cyclohexyl ring or a tetrahydropyranyl ring such as one having one of the formula:

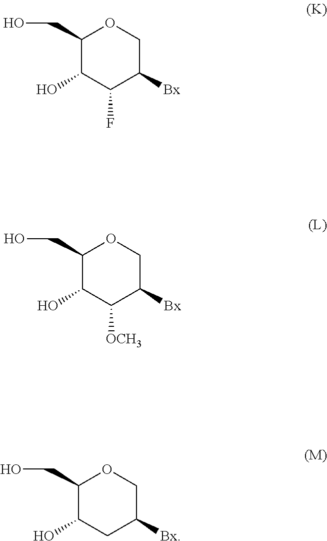

Many other bicyclo and tricyclo sugar surrogate ring systems are also know in the art that can be used to modify nucleosides for incorporation into antisense compounds (see for example review article: Leumann, Christian J., Bioorg. Med. Chem., 2002, 10, 841-854)). Such ring systems can undergo various additional substitutions to enhance activity. See for example compounds having Formula VII:

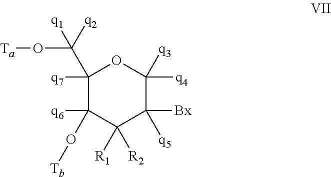

wherein independently for each of said at least one tetrahydropyran nucleoside analog of Formula VII:

Bx is a heterocyclic base moiety;

Tₐ and T_{b} are each, independently, an internucleoside linking group linking the tetrahydropyran nucleoside analog to the antisense compound or one of Tₐ and T_{b} is an internucleoside linking group linking the tetrahydropyran nucleoside analog to the antisense compound and the other of Tₐ and T_{b} is H, a hydroxyl protecting group, a linked conjugate group or a 5′ or 3′-terminal group;

q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl; and each of R₁ and R₂ is selected from hydrogen, hydroxyl, halogen, substituted or unsubstituted alkoxy, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

In certain embodiments, the modified THP nucleosides of Formula VII are provided wherein q₁, q₂, q₃, q₄, q₅, q₆ and q₇ are each H (M). In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, THP nucleosides of Formula VII are provided wherein one of R₁ and R₂ is fluoro (K). In certain embodiments, THP nucleosides of Formula VII are provided wherein one of R₁ and R₂ is methoxyethoxy. In certain embodiments, R₁ is fluoro and R₂ is H; R₁ is H and R₂ is fluoro; R₁ is methoxy and R₂ is H, and R₁ is H and R₂ is methoxyethoxy. Methods for the preparations of modified sugars are well known to those skilled in the art.

In nucleotides having modified sugar moieties, the nucleobase moieties (natural, modified or a combination thereof) are maintained for hybridization with an appropriate nucleic acid target.

In certain embodiments, antisense compounds targeted to a Factor XI nucleic acid comprise one or more nucleotides having modified sugar moieties. In certain embodiments, the modified sugar moiety is 2′-MOE. In certain embodiments, the 2′-MOE modified nucleotides are arranged in a gapmer motif. In certain embodiments, the modified sugar moiety is a bicyclic nucleoside having a (4′-CH(CH₃)—O-2′) bridging group. In certain embodiments, the (4% CH(CH₃)—O-2′) modified nucleotides are arranged throughout the wings of a gapmer motif.

### Modified Nucleobases

Nucleobase (or base) modifications or substitutions are structurally distinguishable from, yet functionally interchangeable with, naturally occurring or synthetic unmodified nucleobases.

Both natural and modified nucleobases are capable of participating in hydrogen bonding. Such nucleobase modifications can impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an antisense compound for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional unmodified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties can also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and O-6 substituted purines, including 2 aminopropyladenine, 5-propynyluracil and 5-propynylcytosine.

In certain embodiments, antisense compounds targeted to a TTC39 nucleic acid comprise one or more modified nucleobases. In certain embodiments, gap-widened antisense oligonucleotides targeted to a TTC39 nucleic acid comprise one or more modified nucleobases. In certain embodiments, the modified nucleobase is 5-methylcytosine. In certain embodiments, each cytosine is a 5-methylcytosine.

### Compositions and Methods for Formulating Pharmaceutical Compositions

Antisense oligonucleotides can be admixed with pharmaceutically acceptable active or inert substance for the preparation of pharmaceutical compositions or formulations. Compositions and methods for the formulation of pharmaceutical compositions are dependent upon a number of criteria, including, but not limited to, route of administration, extent of disease, or dose to be administered.

Antisense compound targeted to a TTC39 nucleic acid can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense compound targeted to a TTC39 nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

### Conjugated Antisense Compounds

Antisense compounds can be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression of TTC39 nucleic acids can be tested in vitro in a variety of cell types. Cell types used for such analyses are available from commercial vendors (e.g. American Type Culture Collection, Manassas, Va.; Zen-Bio, Inc., Research Triangle Park, NC; Clonetics Corporation, Walkersville, Md.) and cells are cultured according to the vendor's instructions using commercially available reagents (e.g. Invitrogen Life Technologies, Carlsbad, Calif.). Illustrative cell types include, but are not limited to, HepG2 cells, Hep3B cells, primary hepatocytes, A549 cells, GM04281 fibroblasts and LLC-MK2 cells.

In Vitro Testing of Antisense Oligonucleotides

Described herein are methods for treatment of cells with antisense oligonucleotides, which can be modified appropriately for treatment with other antisense compounds.

In general, cells are treated with antisense oligonucleotides when the cells reach approximately 60-80% confluence in culture.

One reagent commonly used to introduce antisense oligonucleotides into cultured cells is the cationic lipid transfection reagent LIPOFECTIN® (Invitrogen, Carlsbad, Calif.). Antisense oligonucleotides can be mixed with LIPOFECTIN® in OPTI-MEM® 1 (Invitrogen, Carlsbad, Calif.) to achieve a desired final concentration of antisense oligonucleotide and LIPOFECTIN® concentration that typically ranges 2 to 12 ug/mL per 100 nM antisense oligonucleotide.

Another reagent that can be used to introduce antisense oligonucleotides into cultured cells is LIPOFECTAMINE 2000® (Invitrogen, Carlsbad, Calif.). Antisense oligonucleotide can be mixed with LIPOFECTAMINE 2000® in OPTI-MEM® 1 reduced serum medium (Invitrogen, Carlsbad, Calif.) to achieve a desired concentration of antisense oligonucleotide and LIPOFECTAMINE® concentration that typically ranges 2 to 12 ug/mL per 100 nM antisense oligonucleotide.

Another reagent that can be used to introduce antisense oligonucleotides into cultured cells is Cytofectin® (Invitrogen, Carlsbad, Calif.). Antisense oligonucleotide can be mixed with Cytofectin® in OPTI-MEM® 1 reduced serum medium (Invitrogen, Carlsbad, Calif.) to achieve a desired concentration of antisense oligonucleotide and Cytofectin® concentration that typically ranges 2 to 12 ug/mL per 100 nM antisense oligonucleotide.

Another technique that can be used to introduce antisense oligonucleotides into cultured cells is electroporation.

Cells are treated with antisense oligonucleotides by routine methods. Cells are typically harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE2000®, Lipofectin or Cytofectin. Antisense oligonucleotides are used at higher concentrations ranging from 625 to 20,000 nM when transfected using electroporation.

### RNA Isolation

RNA analysis can be performed on total cellular RNA or poly(A)+ mRNA. Methods of RNA isolation are well known in the art. RNA is prepared using methods well known in the art, for example, using the TRIZOL® Reagent (Invitrogen, Carlsbad, Calif.) according to the manufacturer's recommended protocols.

### Analysis of Inhibition of Target Levels or Expression

Inhibition of levels or expression of a TTC39 nucleic acid can be assayed in a variety of ways known in the art. For example, target nucleic acid levels can be quantitated by, e.g., Southern blot analysis, Northern blot analysis, competitive polymerase chain reaction (PCR), or quantitative real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM® 7600, 7700, or 7900 Sequence Detection System, available from PE-Applied Biosystems, Foster City, Calif. and used according to manufacturer's instructions.

### Quantitative Real-Time PCR Analysis of Target RNA Levels

Quantitation of target RNA levels can be accomplished by quantitative real-time PCR using the ABI PRISMS 7600, 7700, or 7900 Sequence Detection System (PE-Applied Biosystems, Foster City, Calif.) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, Calif.). RT, real-time-PCR reactions are carried out by methods well known to those skilled in the art.

Gene (or RNA) target quantities obtained by real time PCR are normalized using either the expression level of a gene whose expression is constant, such as cyclophilin A or GAPDH, or by quantifying total RNA using RIBOGREEN® (Invitrogen, Inc. Carlsbad, Calif.). Cyclophilin A or GAPDH expression is quantified by real time PCR, by being run simultaneously with the target, multiplexing, or separately. Total RNA is quantified using RIBOGREEN® RNA quantification reagent (Invitrogen, Inc. Eugene, Oreg.). Methods of RNA quantification by RIBOGREEN® are taught in Jones, L. J., et al, (Analytical Biochemistry, 1998, 265, 368-374). A CYTOFLUOR® 4000 instrument (PE Applied Biosystems) is used to measure RIBOGREEN® fluorescence.

Probes and primers are designed to hybridize to a TTC39 nucleic acid. Methods for designing real-time PCR probes and primers are well known in the art, and can include the use of software such as PRIMER EXPRESS® Software (Applied Biosystems, Foster City, Calif.).

### Analysis of Protein Levels

Antisense inhibition of TTC39 nucleic acids can be assessed by measuring TTC39 protein levels. Protein levels of TTC39 can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art.

### In Vivo Testing of Antisense Compounds

Antisense compounds, for example, antisense oligonucleotides, are tested in animals to assess their ability to inhibit expression of TTC39 and produce phenotypic changes. Testing can be performed in normal animals, or in experimental disease models. For administration to animals, antisense oligonucleotides are formulated in a pharmaceutically acceptable diluent, such as phosphate-buffered saline. Administration includes parenteral routes of administration. Following a period of treatment with antisense oligonucleotides, RNA is isolated from tissue and changes in TTC39 nucleic acid expression are measured. Changes in TTC39 protein levels are also measured.

### Certain Indications

In certain embodiments, provided herein are methods of treating an individual comprising administering one or more pharmaceutical compositions as described herein. In certain embodiments, the individual has cardiovascular disease.

Although GWAS has implicated a number of genes in cardiovascular disease or associated genes with markers such as cholesterol levels, follow-up studies in our hands for a number of the implicated genes have not confirmed the association with cardiovascular disease or the markers. However, as shown in the examples below, compounds targeted to TTC39 as described herein have been shown to increase the levels of HDL, LDL receptor, apoA4 and apoA1 and decrease the level PCSK9.

Reverse cholesterol transport (RCT) is a multi-step process resulting in the net movement of cholesterol from peripheral tissues back to the liver via the plasma compartment (Duffy and Rader, Nat Rev Cardiol. 2009 6: 455-63). In the context of atherosclerosis, the removal of excess, proatherogenic cholesterol from an atherosclerotic plaque by RCT is fundamental in mitigating plaque progression and can potentially promote plaque regression. RCT at the plaque site is facilitated by ATP binding cassette (ABC)A1 cellular transporters on macrophages which transfer cholesterol from macrophages to nascent or lipid-poor HDL. The cholesterol is then transported to the liver by HDL where it is selectively removed by scavenger receptor class B Type 1 (SRB1). At the liver, the cholesterol can be converted into bile acid and secreted into the gall bladder or the cholesterol can be directly secreted into bile as biliary cholesterol. RCT is completed when the bile acid or biliary cholesterol is secreted into the intestinal lumen and lost in the feces. Accordingly, RCT is promoted by the interaction of lipid-free or lipid-poor apoA1, the primary protein component of HDL, with ATP binding cassette (ABC)A1 cellular transporters on macrophages.

Apolipoprotein A-IV (apoA4) controls intestinal fat absorption, and has anti-oxidant and anti-atherogenic properties. The apoA4 protein is secreted into the circulation on the surface of chylomicron particles.

Proprotein convertase subtilisin/kexin type 9 (PCSK9) is an enzyme which plays a major regulatory role in cholesterol homeostasis. PCSK9 binds to the low-density lipoprotein receptors (LDLR), inducing LDLR degradation. Reduced LDLR levels result in decreased metabolism of low-density lipoproteins, which could lead to hypercholesterolemia.

Accordingly, provided herein are methods for ameliorating a symptom associated with cardiovascular disease in a subject in need thereof. In certain embodiments, provided is a method for reducing the rate of onset of a symptom associated with cardiovascular disease. In certain embodiments, provided is a method for reducing the severity of a symptom associated with cardiovascular disease. In such embodiments, the methods comprise administering to an individual in need thereof a therapeutically effective amount of a compound targeted to a TTC39 nucleic acid. In certain embodiments, the cardiovascular disease is arteriosclerosis, atherosclerosis, coronary heart disease, heart failure, hypertension, dyslipidemia, hypercholesterolemia, acute coronary syndrome, type II diabetes, type II diabetes with dyslipidemia, hepatic steatosis, non-alcoholic steatohepatitis, non-alcoholic fatty liver disease, hypertriglyceridemia, hyperfattyacidemia, hyperlipidemia and/or metabolic syndrome.

In certain embodiments, administration of an antisense compound targeted to a TTC39 nucleic acid results in reduction of TTC39 expression by at least about 15, 20, 25, 30, 35, 40, 45, 50, 55, 60, 65, 70, 75, 80, 85, 90, 95 or 99%, or a range defined by any two of these values.

In certain embodiments, pharmaceutical compositions comprising an antisense compound targeted to TTC39 are used for the preparation of a medicament for treating a patient suffering or susceptible to cardiovascular disease.

In certain embodiments, the methods described herein include administering a compound comprising a modified oligonucleotide having a contiguous nucleobase portion as described herein complementary to a sequence recited in SEQ ID NO: 1, 15, 2 or 3.

### Administration

In certain embodiments, the compounds and compositions as described herein are administered parenterally.

In certain embodiments, parenteral administration is by infusion. Infusion can be chronic or continuous or short or intermittent. In certain embodiments, infused pharmaceutical agents are delivered with a pump.

In certain embodiments, parenteral administration is by injection. The injection can be delivered with a syringe or a pump. In certain embodiments, the injection is a bolus injection. In certain embodiments, the injection is administered directly to a tissue or organ.

### Certain Combination Therapies

In certain embodiments, a first agent comprising the modified oligonucleotide of the invention is co-administered with one or more secondary agents. In certain embodiments, such second agents are designed to treat the same cardiovascular disease as the first agent described herein. In certain embodiments, such second agents are designed to treat a different disease, disorder, or condition as the first agent described herein. In certain embodiments, such second agents are designed to treat an undesired side effect of one or more pharmaceutical compositions as described herein. In certain embodiments, second agents are co-administered with the first agent to treat an undesired effect of the first agent. In certain embodiments, second agents are co-administered with the first agent to produce a combinational effect. In certain embodiments, second agents are co-administered with the first agent to produce a synergistic effect.

In certain embodiments, a first agent and one or more second agents are administered at the same time. In certain embodiments, the first agent and one or more second agents are administered at different times. In certain embodiments, the first agent and one or more second agents are prepared together in a single pharmaceutical formulation. In certain embodiments, the first agent and one or more second agents are prepared separately.

In certain embodiments, second agents include, but are not limited to statins, ezetamibe, niacin, fibrates, beta blockers, antihypertensives, antithrombotics, inhibitors of TTC39 and inhibitors of PCSK9.

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1

### Antisense Inhibition of Murine TTC39B in Mouse Primary Hepatocytes

Antisense oligonucleotides targeted to a TTC39B nucleic acid were tested for their effects on TTC39B mRNA in vitro. Cultured mouse primary hepatocytes at a density of 10,000 cells per well were transfected using cytofectin reagent with 100 nM antisense oligonucleotide. After a treatment period of approximately 24 hours, RNA was isolated from the cells and TTC39B mRNA levels were measured by quantitative real-time PCR. TTC39B mRNA levels were adjusted according to total RNA content, as measured by RIBOGREEN®. Results are presented as percent inhibition of TTC39B, relative to untreated control cells.

The chimeric antisense oligonucleotides in Tables 1 and 2 were designed as 5-10-5 MOE gapmers. The gapmers are 20 nucleotides in length, wherein the central gap segment is comprised of 10 2′-deoxynucleotides and is flanked on both sides (in the 5′ and 3′ directions) by wings comprising 5 nucleotides each. Each nucleotide in the 5′ wing segment and each nucleotide in the 3′ wing segment has a 2′-MOE modification. The internucleoside linkages throughout each gapmer are phosphorothioate (P═S) linkages. All cytosine residues throughout each gapmer are 5-methylcytosines. “Target start site” indicates the 5′-most nucleotide to which the gapmer is targeted. “Target stop site” indicates the 3′-most nucleotide to which the gapmer is targeted. Each gapmer listed in Table 1 is targeted to SEQ ID NO: 6 (GENBANK Accession No. NM_{—}027238.2) and each gapmer listed in Table 2 is targeted to SEQ ID NO: 10 (the complement of GENBANK Accession No. NT_{—}039260.7 truncated at nucleotides 22491001 to 22601000), SEQ ID NO: 11 (GENBANK Accession No. BY244623.1), SEQ ID NO: 12 (GENBANK Accession No. BY260936), SEQ ID NO: 13 (GENBANK Accession No. BM935568.1) or SEQ ID NO: 14 (GENBANK Accession No. BY728780.1).

‘Mismatches’ indicate the number of nucleobases by which the murine oligonucleotide is mismatched with a human sequence. The designation “n/a” indicates that there was greater than 3 mismatches between the murine oligonucleotide and the human sequence. The greater the complementarity between the murine oligonucleotide and the human sequence, the more likely the murine oligonucleotide can cross-react with the human sequence. The murine oligonucleotides in Table 1 were compared to human mRNA sequence as set forth in GenBank Accession No. NM_{—}152574.1 (SEQ ID NO: 1). The murine oligonucleotides in Table 2 were compared to human genomic sequence as set forth in GenBank Accession No. NT_{—}008413.17, position 15158001 to 15300000 (SEQ ID NO: 15).

TABLE 1
Inhibition of murine TTC39B mRNA levels by chimeric antisense oligonucleotides having 5-10-
5 MOE wings and deoxy gap targeted to SEQ ID NO: 6

| | Target | Target | | | SEQ | |
|---|---|---|---|---|---|---|
| Oligo | Start | Stop | | % | ID | |
| ID | Site | Site | Sequence | inhibition | NO | Mismatches |
| 447098 | 3 | 22 | GGCTCTGGGTTCAGCCCAGC | 44 | 16 | 1 |
| 447099 | 41 | 60 | CCGCACACCCACAGGGAACT | 56 | 17 | n/a |
| 447100 | 84 | 103 | TCCACTCGGCTGCCCACGAG | 62 | 18 | 3 |
| 447101 | 123 | 142 | TCCAAGGCATCTTCAAAGAT | 36 | 19 | 3 |
| 447102 | 148 | 167 | GTCCGATGGAGATCTAGAGA | 84 | 20 | n/a |
| 447103 | 174 | 193 | ACAAAGTGAAAGCCGCTGGT | 64 | 21 | n/a |
| 447104 | 255 | 274 | TCCACCTTGGCTGATGAACC | 48 | 22 | n/a |
| 447105 | 406 | 425 | GACAGCCTGCAACACCACAA | 52 | 23 | 0 |
| 447106 | 441 | 460 | ATGCCATTCTGGATGTCCTG | 70 | 24 | 1 |
| 447107 | 476 | 495 | TTTGGCAGGTTTGTAAAGCG | 61 | 25 | 0 |
| 447108 | 555 | 574 | TCTTCACTCAGTTGTTCCAG | 49 | 26 | 1 |
| 447109 | 635 | 654 | TCATATTTTCATCCTGCACA | 41 | 27 | 0 |
| 447110 | 658 | 677 | GAGTCCACCTTTGATGAAGT | 69 | 28 | 0 |
| 447111 | 695 | 714 | GGCACTCTTTATATATTTGG | 71 | 29 | 2 |
| 447112 | 781 | 800 | CCCCGTTCCAAGCTTGACTC | 62 | 30 | n/a |
| 447113 | 798 | 817 | AGCATCAAATTAAAGGCCCC | 59 | 31 | 0 |
| 447114 | 825 | 844 | CGGATGATTCTTGCTGGTAA | 73 | 32 | 3 |
| 447115 | 857 | 876 | TGTTTCCAGAAAATCCAATA | 56 | 33 | 2 |
| 447116 | 891 | 910 | GCGCCTTCCCGGAGCTGCAG | 56 | 34 | n/a |
| 447117 | 1050 | 1069 | AGTATGAGGGAGCCATTTGG | 47 | 35 | n/a |
| 447118 | 1076 | 1095 | GCAGCTCGATTCGGGCATGA | 76 | 36 | 2 |
| 447119 | 1114 | 1133 | TCGAAATGTTTCTTGAGCCT | 65 | 37 | n/a |
| 447120 | 1182 | 1201 | ATCCACATTAATTCCCAGTA | 65 | 38 | 2 |
| 447121 | 1221 | 1240 | TAGTAATACGCCTGCATCCA | 53 | 39 | 1 |
| 447122 | 1263 | 1282 | GTTGCCTTGGACCATTTACT | 67 | 40 | 0 |
| 447123 | 1284 | 1303 | GCAGCTTTCAAGAACACATA | 47 | 41 | 1 |
| 447124 | 1305 | 1324 | TCTGGAAGCATGCTCAAAAT | 60 | 42 | 1 |
| 447125 | 1486 | 1505 | CATCATTTCCAGGGCTGGCA | 54 | 43 | 2 |
| 447126 | 1595 | 1614 | CAGTAAAGTCTTGATTCTGC | 58 | 44 | n/a |
| 447127 | 1679 | 1698 | GCTCAGCTTGCAAGGGCCGC | 65 | 45 | 1 |
| 447128 | 1712 | 1731 | GTTTTTCACTTTCCACAACG | 56 | 46 | 2 |
| 447129 | 1773 | 1792 | TTATACAAAAATGCCAACTC | 34 | 47 | 2 |
| 447130 | 1809 | 1828 | AGGACCTTGATGGCCTTGTC | 44 | 48 | 2 |
| 447131 | 1892 | 1911 | TCCAGAGATGAAGAGCTGCT | 60 | 49 | 2 |
| 447132 | 1920 | 1939 | GTTCCGGATCAGTCCGTGGA | 58 | 50 | n/a |
| 447133 | 2128 | 2147 | GGCAGAGTATAAGAATTTCT | 56 | 51 | n/a |
| 447134 | 2189 | 2208 | CTTTATTGTACCTGCCAGTC | 53 | 52 | n/a |
| 447135 | 2278 | 2297 | GCCAATCTAAAAATGGCTCA | 67 | 53 | n/a |
| 447136 | 2305 | 2324 | AAAATATCTCTGAAGTTTAA | 24 | 54 | 0 |
| 447137 | 2442 | 2461 | AGATACAAGTCCCTTCTGCC | 50 | 55 | n/a |
| 447138 | 2509 | 2528 | AATGCCACCACCTTCAACCA | 65 | 56 | n/a |
| 447139 | 2541 | 2560 | GAGTACATTCAAATACAGGA | 58 | 57 | n/a |
| 447140 | 2566 | 2585 | TGAATGCTATTATTGGATCT | 66 | 58 | n/a |
| 447141 | 2766 | 2785 | GCAGCCATGGCCCTGGGTCA | 40 | 59 | n/a |
| 447142 | 3073 | 3092 | GGTGGAGTCTGGCCCTCAGC | 66 | 60 | n/a |
| 447143 | 3582 | 3601 | GCCACCAGTGCCTGCTGAGC | 74 | 61 | n/a |
| 447144 | 3713 | 3732 | TATTTCTATAGCTCATGACA | 69 | 62 | n/a |
| 447145 | 3979 | 3998 | TACAGACCATGAGCTTTGAT | 61 | 63 | n/a |
| 447146 | 4121 | 4140 | GGACTGCCTGCCATGGGTGC | 69 | 64 | n/a |
| 447147 | 4434 | 4453 | ACAGTATCTTGAACATGACA | 46 | 65 | n/a |
| 447148 | 4467 | 4486 | TTCTTACAAACATCCTAAGA | 54 | 66 | n/a |
| 447149 | 4978 | 4997 | CCAGACAGACACTCAGACCA | 48 | 67 | n/a |
| 447150 | 5512 | 5531 | CCTGATAAATCACAAATGCT | 46 | 68 | n/a |
| 447151 | 5645 | 5664 | GGAGGTCCCCTGGCCACGGC | 65 | 69 | n/a |
| 447152 | 5795 | 5814 | GACTTTGAGATGGCACACAA | 60 | 70 | n/a |
| 447153 | 7078 | 7097 | GACTTTTAATTCAAAGTCCT | 58 | 71 | n/a |
| 447154 | 8738 | 8757 | CGTGTCATTTATTAAATAAC | 22 | 72 | n/a |

TABLE 2
Inhibition of murine TTC39B mRNA levels by chimeric antisense oligonucleotides having 5-10-
5 MOE wings and deoxy gap targeted to SEQ ID NO: 10, 11, 12, 13 or 14

| | Target | Target | Target | | | | |
|---|---|---|---|---|---|---|---|
| | SEQ ID | Start | Stop | | % | SEQ ID | |
| Oligo ID | NO. | Site | Site | Sequence | inhibition | NO | Mismatches |
| 447158 | 10 | 2428 | 2447 | GGTGCCCGGACCAGCCCGCT | 39 | 73 | n/a |
| 447159 | 10 | 41311 | 41330 | TGTCAGTGACAGTGGCAGCT | 71 | 74 | 2 |
| 447160 | 10 | 41463 | 41482 | AAAGAGGAGTCCTTCCTTAA | 63 | 75 | n/a |
| 447162 | 10 | 47080 | 47099 | AGCCTTGTTCACTGGGAAGA | 59 | 76 | n/a |
| 447163 | 10 | 47185 | 47204 | GCCAGCCAGGGCAGGCACTC | 50 | 77 | n/a |
| 447168 | 10 | 64649 | 64668 | ACTCAGTCATACTTTTGGCA | 35 | 78 | n/a |
| 447169 | 10 | 67007 | 67026 | GAGATCAGTTTCTGATGAAA | 67 | 79 | 3 |
| 447167 | 10 | 67256 | 67275 | GAATTATTTATTCTCACACC | 56 | 80 | n/a |
| 447170 | 10 | 73426 | 73445 | ATCATATTTTCATCCTGAAA | 35 | 81 | 0 |
| 447171 | 10 | 78856 | 78875 | CCTTACCAAATTAAAGGCCC | 23 | 82 | 0 |
| 447172 | 10 | 84173 | 84192 | GTTTCTTGAGCCTATAAGTC | 54 | 83 | n/a |
| 447173 | 10 | 84330 | 84349 | GCTTACCTTGGACCATTTAC | 59 | 84 | 2 |
| 447174 | 10 | 93108 | 93127 | TCCAGATCATTATGTTTACA | 50 | 85 | 0 |
| 447175 | 10 | 93165 | 93184 | TATGGAGAAGCAGATGGCCT | 56 | 86 | 0 |
| 447155 | 11 | 1 | 20 | TCCGGTGCGCACCCAGTACC | 23 | 87 | n/a |
| 447156 | 11 | 79 | 98 | GCCCATGCACGGGTGGAGGA | 16 | 88 | n/a |
| 447157 | 11 | 177 | 196 | TTCCAGCTCGGCTGCGCCAC | 21 | 89 | n/a |
| 447161 | 12 | 258 | 277 | GGAGATGTGTGTACGGCAGC | 14 | 90 | n/a |
| 447164 | 13 | 165 | 184 | TGTCCGATGGAGATTGTTAA | 0 | 91 | n/a |
| 447165 | 13 | 583 | 602 | CGGAGATCAGTTTCTTCACT | 50 | 92 | n/a |
| 447166 | 14 | 57 | 76 | CTGATGAACTTGATTTAGCC | 0 | 93 | n/a |

### Example 2

### Dose-Dependent Antisense Inhibition of Murine TTC39B in Mouse Primary Hepatocytes

Eighteen gapmers, exhibiting 65 percent or greater in vitro inhibition of murine TTC39B (Example 1, Tables 1-2), were further tested at various doses in murine primary hepatocytes. Cells were plated at a density of 10,000 cells per well and transfected using cytofectin reagent with 25 nM, 50 nM, 100 nM, and 200 nM concentrations of antisense oligonucleotide, as specified in Table 3. After a treatment period of approximately 16 hours, RNA was isolated from the cells and TTC39B mRNA levels were measured by quantitative real-time PCR. Murine primer probe set mTtc39b_LTS00287 (forward sequence: CATCTCTAGATCTCCATCGGACATG, incorporated herein as SEQ ID NO: 94; reverse sequence: TGTCTGGAGGTCCGTTTGGT, incorporated herein as SEQ ID NO: 95; probe sequence: CACCAGCGGCTTTCACTTTGTACCATG, incorporated herein as SEQ ID NO: 96) was used to measure mRNA levels. TTC39B mRNA levels were adjusted according to total RNA content, as measured by RIBOGREEN®. Results are presented as percent inhibition of TTC39B, relative to untreated control cells. As illustrated in Table 3, TTC39B mRNA levels were reduced in a dose-dependent manner in antisense oligonucleotide treated cells.

TABLE 3
Dose-dependent antisense inhibition of murine TTC39B mRNA
in mouse primary hepatocytes

| ISIS No. | 25 nM | 50 nM | 100 nM | 200 nM |
|---|---|---|---|---|
| 447106 | 41 | 55 | 78 | 87 |
| 447110 | 39 | 52 | 77 | 87 |
| 447111 | 30 | 60 | 77 | 87 |
| 447114 | 42 | 70 | 85 | 92 |
| 447118 | 49 | 65 | 74 | 82 |
| 447119 | 28 | 52 | 80 | 77 |
| 447122 | 35 | 61 | 78 | 88 |
| 447127 | 40 | 62 | 84 | 90 |
| 447135 | 35 | 52 | 66 | 76 |
| 447138 | 20 | 49 | 68 | 86 |
| 447140 | 29 | 55 | 77 | 80 |
| 447142 | 36 | 64 | 85 | 91 |
| 447143 | 36 | 71 | 79 | 91 |
| 447144 | 37 | 69 | 74 | 84 |
| 447146 | 44 | 55 | 78 | 89 |
| 447151 | 39 | 56 | 76 | 85 |
| 447159 | 41 | 60 | 76 | 85 |
| 447169 | 17 | 43 | 65 | 81 |

### Example 3

### Dose-Dependent Antisense Inhibition of Murine TTC39B in Mouse Primary Hepatocytes

Four gapmers, exhibiting significant dose-dependent inhibition of murine TTC39B (Example 2, Table 3), were further tested at various doses in murine primary hepatocytes. Cells were plated at a density of 10,000 cells per well and transfected using cytofectin reagent with 6.25 nM, 12.5 nM, 25 nM, 50 nM, 100 nM, and 200 nM concentrations of antisense oligonucleotide, as specified in Table 4. After a treatment period of approximately 16 hours, RNA was isolated from the cells and TTC39B mRNA levels were measured by quantitative real-time PCR. Murine primer probe set mTtc39b_LTS00287 was used to measure mRNA levels. TTC39B mRNA levels were adjusted according to total RNA content, as measured by RIBOGREEN®. Results are presented as percent inhibition of TTC39B, relative to untreated control cells. As illustrated in Table 4, TTC39B mRNA levels were reduced in a dose-dependent manner in antisense oligonucleotide treated cells.

TABLE 4
Dose-dependent antisense inhibition of murine TTC39B mRNA
in mouse primary hepatocytes

| | | | | | 100.0 | 200.0 |
|---|---|---|---|---|---|---|
| ISIS No. | 6.25 nM | 12.5 nM | 25.0 nM | 50.0 nM | nM | nM |
| 447118 | 0 | 24 | 45 | 66 | 77 | 81 |
| 447143 | 7 | 25 | 44 | 72 | 88 | 94 |
| 447114 | 6 | 5 | 34 | 71 | 90 | 93 |
| 447159 | 12 | 19 | 37 | 62 | 78 | 86 |

### Example 4

### In Vivo Antisense Inhibition of Murine TTC39B

Four gapmers from the dose-dependent in vitro inhibition study (Example 3) were administered in C57BL/6 mice to evaluate their tolerability and potency.

ISIS 447114 (CGGATGATTCTTGCTGGTAA, incorporated herein as SEQ ID NO: 32) is a chimeric antisense oligonucleotide designed as a 5-10-5 MOE gapmer targeting murine TTC39B (GENBANK Accession No. NM_{—}027238.2, incorporated herein as SEQ ID NO: 6; oligonucleotide target site starting at position 825). The gapmer is 20 nucleotides in length, wherein the central gap segment is comprised of 10 consecutive 2′-deoxynucleosides and is flanked on both sides (in the 5′ and 3′ directions) by wings comprising 5 nucleosides each. Each nucleoside in each wing segment has a 2′-MOE modification. The internucleoside linkages throughout the gapmer are phosphorothioate (P═S) internucleoside linkages. All cytosine residues throughout the gapmer are 5′ methylcytosines.

ISIS 447118 (GCAGCTCGATTCGGGCATGA, incorporated herein as SEQ ID NO: 36) is a chimeric antisense oligonucleotide designed as a 5-10-5 MOE gapmer targeting murine TTC39B (GENBANK Accession No. NM_{—}027238.2, incorporated herein as SEQ ID NO: 6; oligonucleotide target site starting at position 1076). The gapmer is 20 nucleotides in length, wherein the central gap segment is comprised of 10 consecutive 2′-deoxynucleosides and is flanked on both sides (in the 5′ and 3′ directions) by wings comprising 5 nucleosides each. Each nucleoside in each wing segment has a 2′-MOE modification. The internucleoside linkages throughout the gapmer are phosphorothioate (P═S) internucleoside linkages. All cytosine residues throughout the gapmer are 5′ methylcytosines.

ISIS 447143 (GCCACCAGTGCCTGCTGAGC, incorporated herein as SEQ ID NO: 61) is a chimeric antisense oligonucleotide designed as a 5-10-5 MOE gapmer targeting murine TTC39B (GENBANK Accession No. NM_{—}027238.2, incorporated herein as SEQ ID NO: 6; oligonucleotide target site starting at position 3582). The gapmer is 20 nucleotides in length, wherein the central gap segment is comprised of 10 consecutive 2′-deoxynucleosides and is flanked on both sides (in the 5′ and 3′ directions) by wings comprising 5 nucleosides each. Each nucleoside in each wing segment has a 2′-MOE modification. The internucleoside linkages throughout the gapmer are phosphorothioate (P═S) internucleoside linkages. All cytosine residues throughout the gapmer are 5′ methylcytosines.

ISIS 447144 (TATTTCTATAGCTCATGACA, incorporated herein as SEQ ID NO: 62) is a chimeric antisense oligonucleotide designed as a 5-10-5 MOE gapmer targeting murine TTC39B (GENBANK Accession No. NM_{—}027238.2, incorporated herein as SEQ ID NO: 6; oligonucleotide target site starting at position 3713). The gapmer is 20 nucleotides in length, wherein the central gap segment is comprised of 10 consecutive 2′-deoxynucleosides and is flanked on both sides (in the 5′ and 3′ directions) by wings comprising 5 nucleosides each. Each nucleoside in each wing segment has a 2′-MOE modification. The internucleoside linkages throughout the gapmer are phosphorothioate (P═S) internucleoside linkages. All cytosine residues throughout the gapmer are 5′ methylcytosines.

Eight week old male C57BL/6 mice were obtained from The Jackson Laboratory (Bar Harbor, Me.).

Treatment

Four groups of five mice each were injected twice a week with 25 mg/kg (totaling 50 mg/kg/week) of ISIS 447114, ISIS 447118, ISIS 447143, or ISIS 447144 for 6 weeks. A control group of five mice was injected with phosphate buffered saline (PBS) twice a week for 6 weeks. Plasma samples were obtained before the start of treatment, at week 3 and at week 6. At the end of the treatment period, mice were fasted for 4 hours before being euthanized. Plasma and tissue samples were obtained for further analysis.

RNA Analysis

RNA was extracted from liver tissue for real-time PCR analysis of TTC39B using murine primer probe set mTtc39b_LTS00287. As shown in Table 5, the antisense oligonucleotides achieved significant reduction of murine TTC39B over the PBS control. Results are presented as an average of the percent inhibition of TTC39B, relative to control.

TABLE 5
In vivo antisense inhibition of murine TTC39B mRNA in C57BL/6 mice

| ISIS No | % inhibition |
|---|---|
| 447114 | 49 |
| 447118 | 73 |
| 447143 | 85 |
| 447144 | 70 |

Effect of Antisense Inhibition of TTC39B on Proteins Involved in Reverse Cholesterol Transport

Reverse cholesterol transport is a multi-step process resulting in the net movement of cholesterol from peripheral tissues back to the liver via the plasma compartment (Duffy and Rader, Nat Rev Cardiol. 2009 6: 455-63). In the context of atherosclerosis, the removal of excess, proatherogenic cholesterol from an atherosclerotic plaque by RCT is fundamental in mitigating plaque progression and can potentially promote plaque regression. RCT at the plaque site is facilitated by ATP binding cassette (ABC)A1 cellular transporters on macrophages which transfer cholesterol from macrophages to nascent or lipid-poor HDL. The cholesterol is then transported to the liver by HDL where it is selectively removed by scavenger receptor class B Type 1 (SRB1). At the liver, the cholesterol can be converted into bile acid and secreted into the gall bladder or the cholesterol can be directly secreted into bile as biliary cholesterol. RCT is completed when the bile acid or biliary cholesterol is secreted into the intestinal lumen and lost in the feces. Accordingly, RCT is promoted by the interaction of lipid-free or lipid-poor apoA1, the primary protein component of HDL, with ATP binding cassette (ABC)A1 cellular transporters on macrophages.

The effect of inhibition of TTC39B in mice on these proteins of reverse cholesterol transport was studied. RNA levels of ABCA1 were measured using murine primer probe set RTS1204 (forward sequence GGACTTGGTAGGACGGAACCT, designated herein as SEQ ID NO: 97; reverse sequence ATCCTCATCCTCGTCATTCAAAG, designated herein as SEQ ID NO: 98; and probe sequence AGGCCCAGACCTGTAAAGGCGAAG, designated herein as SEQ ID NO: 99). RNA levels of apoA1 were measured using murine primer probe set RTS14737 (forward sequence ACTCTGGGTTCAACCGTTAGTCA, designated herein as SEQ ID NO: 100; reverse sequence TATCCCAGAAGTCCCGAGTCAA, designated herein as SEQ ID NO: 101; and probe sequence CTGCAGGAACGGCTGGGCCC, designated herein as SEQ ID NO: 102). RNA levels of SRB1 were measured using murine primer probe set mSRB-1 (forward sequence TGACAACGACACCGTGTCCT, designated herein as SEQ ID NO: 103; reverse sequence ATGCGACTTGTCAGGCTGG, designated herein as SEQ ID NO: 104; and probe sequence CGTGGAGAACCGCAGCCTCCATT, designated herein as SEQ ID NO: 105).

As presented in Table 6, inhibition of TTC39B by ISIS 447143 and ISIS 447144 led to an average increase in apoA1 mRNA levels. The average levels of ABCA1 and SRB1 were modestly decreased or unaffected by treatment by any of the ISIS oligonucleotides.

TABLE 6
Effect of antisense inhibition of murine TTC39B mRNA on reverse
cholesterol transport protein mRNA levels.

| ISIS No | apoA1 | abcA1 | srb1 |
|---|---|---|---|
| 447114 | −14 | −18 | −12 |
| 447118 | −23 | −2 | 0 |
| 447143 | +1 | −17 | 0 |
| 447144 | +50 | 0 | 0 |

The increase in apoA1 mRNA levels was further corroborated with average increases seen in APOA1 plasma protein levels. As presented in Table 7, there was an average increase of APOA1 by 61% over the PBS control at week 6.

TABLE 7
Effect of antisense inhibition on APOA1 plasma protein levels

| | | | | % change |
|---|---|---|---|---|
| | | | | over the |
| | Week 0 | Week 3 | Week 6 | baseline |
| PBS | +10 | +13 | +14 | +46 |
| ISIS 447114 | +9 | +12 | +22 | +122 |
| ISIS 447118 | +9 | +13 | +18 | +88 |
| ISIS 447143 | +10 | +15 | +20 | +108 |
| ISIS 447144 | +10 | +14 | +20 | +108 |

Effect of Antisense Inhibition of TTC39B on Proteins Involved in Cholesterol Homeostasis.

Proprotein convertase subtilisin/kexin type 9, also known as PCSK9, is an enzyme which plays a major regulatory role in cholesterol homeostasis. PCSK9 binds to the low-density lipoprotein receptors (LDLR), inducing LDLR degradation. Reduced LDLR levels result in decreased metabolism of low-density lipoproteins, which could lead to hypercholesterolemia.

The effect of inhibition of TTC39B in mice on cholesterol homeostasis was studied. PCSK9 mRNA levels were measured using the murine primer probe set mPCSK9 (forward sequence ACCGACTTCAACAGCGTGC, designated herein as SEQ ID NO: 106; reverse sequence GGCTGTCACACTTGCTCGC, designated herein as SEQ ID NO: 107, probe sequence AGGATGGGACACGCTTCCACAGACAX, wherein X is a fluorophore, designated herein as SEQ ID NO: 108). LDLR mRNA levels were measured using the murine primer probe set mLDLR (forward sequence GACCGCAGCGAGTACACCA, designated herein as SEQ ID NO: 109; reverse sequence TCACCTCCGTGTCGAGAGC, designated herein as SEQ ID NO: 110, probe sequence TCTGCTCCCCAACCTGAAGAATGTGGT, designated herein as SEQ ID NO: 111). The results are presented as percentage inhibition compared to the PBS control.

As presented in Table 8, treatment with ISIS oligonucleotides targeting TTC39B reduced PCSK9 mRNA levels. LDLR mRNA levels did not display any significant change. These findings suggest that antisense inhibition of TTC39B may have a significant effect in lowering cholesterol in patients with hypercholesterolemia.

TABLE 8
Percent inhibition PCSK9 and LDLR mRNA levels

| ISIS No. | PCSK9 | LDLR |
|---|---|---|
| 447114 | 24 | 2 |
| 447118 | 45 | 6 |
| 447143 | 65 | 34 |
| 447144 | 19 | 2 |

Effect of Antisense Inhibition of TTC39B on Lipids

Cellular cholesterol efflux is mediated by HDL; low levels of HDL cholesterol are a significant predictor of atherosclerotic cardiovascular events. Plasma levels of HDL and apolipoprotein A1 (apoA1) are inversely correlated with the risk of cardiovascular disease (Caveliar et al, Biochim Biophys Acta. 2006 1761: 655-66).

To investigate the effect of inhibition of TTC39B on triglycerides and cholesterol levels in the plasma, samples were collected on weeks 0, 3, and 6, and total plasma cholesterol, LDL cholesterol, HDL cholesterol, and triglycerides were analyzed on an Olympus AU400e Analyzer.

Inhibition of TTC39B levels resulted in significant increases of HDL cholesterol levels starting from week 4. The results are presented in Table 9 as an average expressed in mg/dL. Treatment with ISIS oligonucleotides caused increases in HDL cholesterol levels by an average of 103% over the levels taken before the start of treatment, and this was over 50% of the PBS control at week 6 (Table 10), suggesting that treatment with ISIS oligonucleotides can significantly alter HDL cholesterol levels.

TABLE 9
Effect of antisense inhibition on HDL cholesterol levels (mg/dL)

| | Week 0 | Week 3 | Week 6 |
|---|---|---|---|
| PBS | 44 | 47 | 63 |
| ISIS 447114 | 42 | 47 | 96 |
| ISIS 447118 | 45 | 50 | 100 |
| ISIS 447143 | 44 | 56 | 91 |
| ISIS 447144 | 46 | 53 | 90 |

TABLE 10
Effect of antisense inhibition on HDL cholesterol levels
(% change over baseline)

| | Week 3 | Week 6 |
|---|---|---|
| PBS | +8 | +52 |
| ISIS 447114 | +14 | +130 |
| ISIS 447118 | +15 | +89 |
| ISIS 447143 | +28 | +104 |
| ISIS 447144 | +14 | +88 |

The levels of total cholesterol increased as a result of increases in HDL cholesterol levels by an average of 44% over the PBS control at week 6 (Table 11). The percentage change over the baseline average of 72 mg/dL is also presented. The levels of LDL cholesterol and triglycerides did not change significantly with treatment (Tables 12 and 13).

TABLE 11
Effect of antisense inhibition on total cholesterol levels (mg/dL)

| | | | | % increase |
|---|---|---|---|---|
| | | | | over |
| | Week 0 | Week 3 | Week 6 | baseline |
| PBS | 72 | 69 | 82 | +15 |
| ISIS 447114 | 66 | 70 | 114 | +61 |
| ISIS 447118 | 71 | 77 | 97 | +37 |
| ISIS 447143 | 70 | 81 | 104 | +46 |
| ISIS 447144 | 75 | 81 | 107 | +37 |

TABLE 12
Effect of antisense inhibition on LDL cholesterol levels (mg/dL)

| | Week 0 | Week 3 | Week 6 |
|---|---|---|---|
| PBS | 13 | 12 | 14 |
| ISIS 447114 | 11 | 12 | 19 |
| ISIS 447118 | 14 | 13 | 15 |
| ISIS 447143 | 13 | 14 | 17 |
| ISIS 447144 | 14 | 14 | 18 |

TABLE 13
Effect of antisense inhibition on triglyceride levels (mg/dL)

| | Week 0 | Week 3 | Week 6 |
|---|---|---|---|
| PBS | 87 | 66 | 92 |
| ISIS 447114 | 107 | 66 | 113 |
| ISIS 447118 | 82 | 69 | 88 |
| ISIS 447143 | 95 | 92 | 95 |
| ISIS 447144 | 78 | 70 | 91 |

In general, wildtype lean mice do not develop cardiovascular disease and the largest portion of their cholesterol is found in HDL. Therefore, significantly raising the HDL levels in these mice is difficult. The studies herein show that antisense oligonucleotide inhibition of TTC39B promotes unexpectedly vigorous increases in the HDL cholesterol levels of the wildtype lean mice.

Accordingly, treatment with ISIS oligonucleotides targeting TTC39B would be beneficial for patients with cardiovascular disorders, such as dyslipidemia and hypercholesterolemia.

Effect of Antisense Inhibition of TTC39B on Glucose Levels

Plasma glucose values were determined using a Beckman Glucose Analyzer II (Beckman Coulter) by a glucose oxidase assay. As presented in Table 14, there was no significant change in glucose levels after treatment with ISIS oligonucleotides.

TABLE 14
Effect of antisense inhibition on glucose levels (mg/dL)

| | Week 0 | Week 3 | Week 6 |
|---|---|---|---|
| PBS | 266 | 258 | 262 |
| ISIS 447114 | 293 | 229 | 232 |
| ISIS 447118 | 242 | 271 | 238 |
| ISIS 447143 | 238 | 231 | 241 |
| ISIS 447144 | 222 | 239 | 235 |

Effect of antisense inhibition of TTC39B on free fatty acid levels Plasma levels of non-esterified fatty acids (NEFA) and 3-hydroxybutyric acid (3-HB), which are the end-products of fatty acid oxidation, were measured using an automated clinical chemistry analyzer (Olympus Clinical Analyzer). The results are presented in Tables 15 and 16, the average is expressed in mEq/L. Treatment with ISIS oligonucleotide resulted in an average increase in free fatty acid levels in the plasma by 78% over the PBS control at week 6. Levels of 3-HB were not significantly affected by treatment.

TABLE 15
Effect of antisense inhibition on NEFA levels

| | | | | % change |
|---|---|---|---|---|
| | | | | over the |
| | Week 0 | Week 3 | Week 6 | baseline |
| PBS | 0.50 | 0.43 | 0.83 | +33 |
| ISIS 447114 | 0.67 | 0.49 | 1.52 | +144 |
| ISIS 447118 | 0.67 | 0.51 | 1.18 | +90 |
| ISIS 447143 | 0.70 | 0.72 | 1.30 | +109 |
| ISIS 447144 | 0.57 | 0.63 | 1.25 | +101 |

TABLE 16
Effect of antisense inhibition on 3-HB levels

| | | | | % change |
|---|---|---|---|---|
| | | | | over the |
| | Week 0 | Week 3 | Week 6 | baseline |
| PBS | 253 | 191 | 351 | +122 |
| ISIS 447114 | 145 | 109 | 449 | +184 |
| ISIS 447118 | 158 | 111 | 406 | +157 |
| ISIS 447143 | 108 | 89 | 222 | +41 |
| ISIS 447144 | 124 | 122 | 398 | +152 |

Effect of Antisense Inhibition of TTC39B on Body and Organ Weights

Body weights of all the mice were measured weekly till the end of the treatment period. Organ weights were taken at the end of the treatment period when the mice were euthanized. The results are presented in Tables 17 and 18, and indicate that treatment with ISIS oligonucleotides did not cause any adverse changes in the health of the mice, as indicated by changes in weights.

TABLE 17
Weekly body weight measurements (g) during treatment

| | week 1 | week 2 | week 3 | week 4 | week 5 | week 6 |
|---|---|---|---|---|---|---|
| PBS | 23 | 24 | 25 | 25 | 26 | 27 |
| ISIS 447114 | 25 | 25 | 26 | 26 | 27 | 27 |
| ISIS 447118 | 24 | 25 | 26 | 26 | 27 | 27 |
| ISIS 447143 | 26 | 26 | 27 | 27 | 28 | 28 |
| ISIS 447144 | 25 | 26 | 26 | 27 | 27 | 28 |

TABLE 18
Organ weight measurements (g) after treatment

| | Liver | Kidney | Spleen |
|---|---|---|---|
| PBS | 1.2 | 0.3 | 0.08 |
| ISIS 447114 | 1.3 | 0.33 | 0.09 |
| ISIS 447118 | 1.4 | 0.32 | 0.08 |
| ISIS 447143 | 1.5 | 0.34 | 0.08 |
| ISIS 447144 | 1.3 | 0.34 | 0.08 |

Evaluation of Liver Function

To evaluate the impact of ISIS oligonucleotides on the hepatic function of the mice described above, plasma concentrations of transaminases were measured using an automated clinical chemistry analyzer (Olympus Clinical Analyzer). Measurements of alanine transaminase (ALT) and aspartate transaminase (AST) are expressed in IU/L. The results are presented in Tables 19 and 20 and indicate that the oligonucleotides were well tolerated.

TABLE 19
Effect of antisense oligonucleotide treatment on ALT levels (IU/L)

| | Week 0 | Week 3 | Week 6 |
|---|---|---|---|
| PBS | 44 | 29 | 36 |
| ISIS 447114 | 46 | 21 | 40 |
| ISIS 447118 | 29 | 39 | 50 |
| ISIS 447143 | 32 | 31 | 45 |
| ISIS 447144 | 28 | 21 | 69 |

TABLE 20
Effect of antisense oligonucleotide treatment on AST levels (IU/L)

| | Week 0 | Week 3 | Week 6 |
|---|---|---|---|
| PBS | 229 | 37 | 148 |
| ISIS 447114 | 83 | 29 | 93 |
| ISIS 447118 | 88 | 39 | 127 |
| ISIS 447143 | 50 | 31 | 73 |
| ISIS 447144 | 67 | 31 | 113 |

### Example 5

### Effect of Antisense Inhibition of Murine TTC39B in the LDLr^{-/-} Mice Model

ISIS 447118, which displayed high potency and tolerability in vivo (Example 4), was administered in the LDL receptor knockout mice model to evaluate its tolerability, potency and effect on hypercholesterolemia and atherosclerosis in this model.

The LDLr knockout mice model is a model for familial hypercholesterolemia developed by Ishibashi et al (J Clin Invest 1993; 92: 883-893). On a normal chow diet, the mice have an average total cholesterol of 250 mg/dL due to increases in LDL and VLDL cholesterol. This model is therefore highly susceptible to atherosclerosis (Ishibashi et al Proc Natl Acad Sci USA 1994; 91: 4431-4435).

Treatment

Four groups of five male mice each were injected with a total of 1.5, 5, 15, or 50 mg/kg/week of ISIS 447118 for 6 weeks. A group of five mice was injected with a total of 50 mg/kg/week of control oligonucleotide ISIS 141923 (CCTTCCCTGAAGGTTCCTCC, designated herein as SEQ ID NO: 112, which has no known murine target) for 6 weeks. A control group of five mice was injected with phosphate buffered saline (PBS) twice a week for 6 weeks. Plasma samples were obtained before the start of treatment, at week 3 and at week 6. At the end of the treatment period, the mice were euthanized. Plasma and tissue samples were obtained for further analysis.

Effect of Antisense Inhibition on Cholesterol and Triglyceride Levels

Cellular cholesterol efflux is mediated by HDL; low levels of HDL cholesterol are a significant predictor of atherosclerotic cardiovascular events.

To investigate the effect of inhibition of TTC39B on triglycerides and cholesterol levels in the plasma, samples were collected on weeks 0, 3, and 6, and total plasma cholesterol, LDL cholesterol, HDL cholesterol, and triglycerides were analyzed on an Olympus AU400e Analyzer.

Inhibition of TTC39B levels resulted in significant increases of HDL cholesterol levels on week 6 with all four doses. The results are presented in Table 21 as an average expressed in mg/dL. Treatment with the ISIS oligonucleotide at 50 mg/kg/wk caused increases in HDL cholesterol levels by an average of 58% over the levels taken before the start of treatment, and this was over 22% of the PBS control at week 6 (Table 22), suggesting that treatment with ISIS oligonucleotides can significantly increase HDL cholesterol levels.

TABLE 21
Effect of antisense inhibition on HDL cholesterol levels (mg/dL)

| | Dose | Week 0 | Week 3 | Week 6 |
|---|---|---|---|---|
| PBS | — | 76 | 85 | 103 |
| ISIS 141923 | 50 | 76 | 78 | 108 |
| ISIS 447118 | 1.5 | 75 | 71 | 101 |
| | 5 | 88 | 83 | 112 |
| | 15 | 84 | 82 | 122 |
| | 50 | 78 | 85 | 124 |

TABLE 22
Effect of antisense inhibition on HDL cholesterol levels
(% change over baseline)

| | | % increase on |
|---|---|---|
| | Dose | week 6 |
| PBS | | 36 |
| ISIS 141923 | 50 | 43 |
| ISIS 447118 | 1.5 | 36 |
| | 5 | 28 |
| | 15 | 45 |
| | 50 | 58 |

The levels of total cholesterol, LDL cholesterol and triglycerides are shown in Tables 23, 24 and 25, respectively. The increase in total cholesterol may be a result of the increase in HDL.

TABLE 23
Effect of antisense inhibition on total cholesterol levels (mg/dL)

| | Dose | Week 0 | Week 3 | Week 6 |
|---|---|---|---|---|
| PBS | | 222 | 216 | 229 |
| ISIS 141923 | 50 | 206 | 204 | 227 |
| ISIS 447118 | 1.5 | 227 | 217 | 247 |
| | 5 | 234 | 232 | 261 |
| | 15 | 239 | 249 | 294 |
| | 50 | 207 | 224 | 282 |

TABLE 24
Effect of antisense inhibition on LDL cholesterol levels (mg/dL)

| | Dose | Week 0 | Week 3 | Week 6 |
|---|---|---|---|---|
| PBS | | 113 | 103 | 117 |
| ISIS 141923 | 50 | 100 | 100 | 112 |
| ISIS 447118 | 1.5 | 115 | 116 | 131 |
| | 5 | 112 | 120 | 130 |
| | 15 | 122 | 133 | 158 |
| | 50 | 99 | 111 | 146 |

TABLE 25
Effect of antisense inhibition on triglyceride levels (mg/dL)

| | Dose | Week 0 | Week 3 | Week 6 |
|---|---|---|---|---|
| PBS | | 113 | 117 | 128 |
| ISIS 141923 | 50 | 97 | 116 | 143 |
| ISIS 447118 | 1.5 | 126 | 107 | 142 |
| | 5 | 118 | 122 | 149 |
| | 15 | 120 | 126 | 131 |
| | 50 | 98 | 96 | 115 |

The studies herein show that antisense oligonucleotide inhibition of TTC39B in LDLr knockout mice promotes unexpectedly vigorous increases in the HDL cholesterol levels.

Accordingly, treatment with ISIS oligonucleotides targeting TTC39B could be beneficial for patients with cardiovascular disorders, such as dyslipidemia, atherosclerosis and hypercholesterolemia.

PCR Microarray Analysis

Liver samples from the mice groups were analyzed using a commercial kit for PCR microarray analysis (SA Biosciences Corp., MD), according to the manufacturer's instructions. Genes involved in lipoprotein and cholesterol metabolism were compared with the PBS control. The results are presented in Table 26.

As presented in Table 26, antisense inhibition of TTC39B resulted in an increase in apoA4 mRNA, which controls intestinal fat absorption, and has anti-atherogenic properties (Duverger et al, Science 273 (5277): 966-8). PCSK9 mRNA was significantly decreased, similar to the observations in the C57BL/6 model (Example 4). Therefore, antisense inhibition of TTC39B resulted in significant changes in several genes involved in lipid and cholesterol metabolism. Hence treatment with ISIS oligonucleotides could be beneficial for individuals suffering from cardiovascular disorders such as dyslipidemia, atherosclerosis and hypercholesterolemia

TABLE 26
Fold increase/decrease relative to PBS group

| | | ISIS 141923 | 447118 | 447118 | 447118 | 447118 |
|---|---|---|---|---|---|---|
| Gene Name | Symbol | (50 mg/kg) | (1.5 mg/kg) | (5 mg/kg) | (15 mg/kg) | (50 mg/kg) |
| Apolipoprotein A-IV | ApoA4 | 0.87 | 1.26 | 1.38 | 1.76 | 2.7 |
| Low density | Ldlr | 1.36 | 1.43 | 1.28 | 1.79 | 2.87 |
| lipoprotein receptor | | | | | | |
| Oxysterol binding | Osbpl5 | 1.21 | 1.7 | 2.34 | 3.11 | 2.11 |
| protein-like 5 | | | | | | |
| Sterol O- | Soat2 | 0.95 | 1.05 | 0.88 | 1.36 | 1.79 |
| acyltransferase 2 | | | | | | |
| Stabilin 1 | Stab1 | 1.12 | 1.15 | 1.19 | 1.35 | 1.53 |
| Farnesyl diphosphate | Fdps | 1.21 | 1.03 | 0.84 | 0.8 | 0.66 |
| synthetase | | | | | | |
| Insulin induced gene 1 | Insig1 | 1.13 | 0.96 | 0.84 | 0.68 | 0.67 |
| Mevalonate | Mvd | 1.25 | 0.81 | 0.7 | 0.56 | 0.67 |
| (diphospho) | | | | | | |
| decarboxylase | | | | | | |
| Proprotein convertase | Pcsk9 | 1.09 | 0.83 | 0.66 | 0.4 | 0.23 |
| subtilisin/kexin type 9 | | | | | | |

It is expected that antisense oligonucleotides targeted to TTC39A and TTC39C will provide similar beneficial effects to those provided by oligonucleotides targeted to TTC39B, as tested in Examples 1-5 herein. Antisense oligonucleotides targeted to TTC39A and TTC39C are expected to behave similarly to antisense oligonucleotides targeted to TTC39B because TTC39A and TTC39C are isoforms of TTC39B with each TTC39 isoform having at least one tetratricopeptide repeat (TPR) motif consisting of two antiparallel α-helices. Antisense oligonucleotides targeted to TTC39A and TTC39C are therefore expected to increase HDL levels, increase LDLr levels, increase apoA1 levels and increase apoA4 levels while decreasing PCSK9 levels.

### Example 6

### Antisense Inhibition of Mouse Tetratricopeptide Repeat Domain 39A (TTC39A) in b.END Cells

Antisense oligonucleotides were designed targeting a TTC39A nucleic acid and were tested for their effects on TTC39A mRNA in vitro. Cultured b.END cells at a density of 4,000 cells per well were transfected using cytofectin reagent with 70 nM antisense oligonucleotide. After a treatment period of approximately 24 hours, RNA was isolated from the cells and TTC39A mRNA levels were measured by quantitative real-time PCR. Mouse primer probe set RTS3262 (forward sequence TGTGCGTCATGCTGTTGCT, designated herein as SEQ ID NO: 113; reverse sequence CCTCGATGTTGACATTCCCAGTA, designated herein as SEQ ID NO: 114; probe sequence TGTTATCACACCTTCCTCACCTTCGTGCTC, designated herein as SEQ ID NO: 115). TTC39A mRNA levels were adjusted according to total RNA content, as measured by RIBOGREEN®. Results are presented as percent inhibition of TTC39A, relative to untreated control cells.

The chimeric antisense oligonucleotides targeting TTC39A were designed as 5-10-5 MOE gapmers. The gapmers are 20 nucleotides in length, wherein the central gap segment is comprised of ten 2′-deoxynucleotides and is flanked on both sides (in the 5′ and 3′ directions) by wings comprising five nucleotides each. Each nucleotide in the 5′ wing segment and each nucleotide in the 3′ wing segment has a 2′-MOE modification. The internucleoside linkages throughout each gapmer are phosphorothioate (P═S) linkages. All cytosine residues throughout each gapmer are 5-methylcytosines. “Mouse Target start site” indicates the 5′-most nucleotide to which the gapmer is targeted in a mouse nucleic acid sequence.

Over a hundred chimeric antisense oligonucleotides were designed to target at least one of the following murine TTC39A sequences: a mRNA sequence, designated herein as SEQ ID NO: 116 (GENBANK Accession No. NM_{—}153392.2), a genomic sequence, designated herein as SEQ ID NO: 117 (GENBANK Accession No. NT_{—}039264.6 truncated from nucleotides 9527001 to 9571000), SEQ ID NO: 118 (GENBANK Accession No. BF182232.1), SEQ ID NO: 119 (GENBANK Accession No. BF540348.1), SEQ ID NO: 120 (GENBANK Accession No. BG861605.1), SEQ ID NO: 121 (GENBANK Accession No. BG862399.1), SEQ ID NO: 122 (GENBANK Accession No. BI082980.1), SEQ ID NO: 123 (GENBANK Accession No. BI147002.1), SEQ ID NO: 124 (the complement of GENBANK Accession No. BY024857.1), SEQ ID NO: 125 (GENBANK Accession No. CF915889.1), SEQ ID NO: 126 (GENBANK Accession No. NM_{—}001145948.1), SEQ ID NO: 127 (GENBANK Accession No. W89566.1) or SEQ ID NO: 128 (GENBANK Accession No. BX513784.1). Table 27 shows an exemplary selection of eleven chimeric antisense oligonucleotides targeting TTC39A that exhibited good inhibition of TTC39A.

The murine oligonucleotides of Table 27 may also be cross-reactive with human mRNA sequence. ‘Mismatches’ indicate the number of nucleobases by which the murine oligonucleotide is mismatched with a human mRNA sequence. The greater the complementarity between the murine oligonucleotide and the human mRNA sequence, the more likely the murine oligonucleotide can cross-react with the human sequence. The murine oligonucleotides in Table 27 were compared to SEQ ID NO: 129 (GenBank Accession No. NM_{—}001144832.1). “Human Target start site” indicates the 5′-most nucleotide to which the antisense oligonucleotide is targeted in the human sequence.

TABLE 27
Inhibition of murine TTC39A mRNA levels by chimeric antisense oligonucleotides having 5-10-
5 MOE wings and deoxy gap targeted to various TTC39A sequences

| Mouse | Mouse | | | | Human | | SEQ |
|---|---|---|---|---|---|---|---|
| Start | Target | | | % | start | | ID |
| Site | SEQ ID | ISIS No | Sequence | inhibition | Site | Mismatches | NO |
| 132 | 116 | 474351 | TGCACTGGTCCAGGGCCTCA | 94 | 145 | 0 | 130 |
| 1273 | 116 | 474369 | CGCCTGGACTTCCGGATAGC | 96 | 1286 | 2 | 131 |
| 2175 | 116 | 474382 | GCATGTAGAGAACTTCCAGG | 95 | n/a | n/a | 132 |
| 12879 | 117 | 474498 | CACCTGGGTTTGAGGTAGCT | 95 | n/a | n/a | 133 |
| 29919 | 117 | 474366 | TCCTTGCTCAGCAGGTCGGC | 93 | 1100 | 1 | 134 |
| 41381 | 117 | 474382 | GCATGTAGAGAACTTCCAGG | 95 | n/a | n/a | 132 |
| 319 | 125 | 474474 | TCGGTGCCTCTGACACAGCG | 97 | 348 | 2 | 135 |
| 25705 | 117 | 474437 | GCCTCAGAATCCTGGTGGGA | 94 | 685 | 3 | 136 |
| 4167 | 117 | 474497 | AGTTGGGACCCAGAAGGCCT | 94 | n/a | n/a | 137 |
| 518 | 116 | 474435 | CCGCACTTTGATGCCGCCCT | 94 | 531 | 3 | 138 |
| 1687 | 116 | 474452 | TCCATGGAGTAGTTCTTGTA | 92 | 1700 | 1 | 139 |

### Example 7

### Dose-Dependent Antisense Inhibition of Murine TTC39A in b.END Cells

Eleven antisense oligonucleotides, exhibiting 90 percent or greater in vitro inhibition of murine TTC39A (Example 6, Table 27), were further tested at various doses in b.END cells. Cells were plated at a density of 3,500 cells per well and transfected using cytofectin reagent with 4.375 nM, 8.75 nM, 17.5 nM, 35 nM, 70 nM, and 140 nM concentrations of antisense oligonucleotide, as specified in Table 28. After a treatment period of approximately 16 hours, RNA was isolated from the cells and TTC39A mRNA levels were measured by quantitative real-time PCR. Murine primer probe set RTS3262 was used to measure mRNA levels. TTC39A mRNA levels were adjusted according to total RNA content, as measured by RIBOGREEN®. Results are presented as percent inhibition of TTC39A, relative to untreated control cells. As illustrated in Table 28, TTC39A mRNA levels were reduced in a dose-dependent manner in antisense oligonucleotide treated cells.

TABLE 28
Dose-dependent antisense inhibition of murine TTC39A
mRNA in b.END cells

| ISIS | 4.375 | | 17.5 | | | 140.0 | IC₅₀ |
|---|---|---|---|---|---|---|---|
| No | nM | 8.75 nM | nM | 35.0 nM | 70.0 nM | nM | (nM) |
| 474369 | 0 | 14 | 22 | 48 | 62 | 82 | 44 |
| 474382 | 0 | 10 | 24 | 41 | 68 | 80 | 42 |
| 474351 | 6 | 0 | 8 | 48 | 66 | 84 | 44 |
| 474366 | 0 | 0 | 23 | 45 | 64 | 80 | 43 |
| 474380 | 10 | 0 | 4 | 37 | 46 | 73 | 76 |
| 474474 | 0 | 5 | 27 | 56 | 72 | 87 | 33 |
| 474498 | 0 | 6 | 33 | 55 | 64 | 80 | 35 |
| 474437 | 5 | 6 | 26 | 36 | 62 | 82 | 49 |
| 474497 | 8 | 5 | 21 | 29 | 53 | 77 | 72 |
| 474435 | 0 | 2 | 11 | 8 | 52 | 76 | 67 |
| 474452 | 0 | 9 | 36 | 46 | 61 | 86 | 41 |

### Example 8

### Antisense Inhibition of Mouse Tetratricopeptide Repeat Domain 39C (TTC39C) in Mouse Primary Hepatocytes

Antisense oligonucleotides were designed targeting a TTC39C nucleic acid and were tested for their effects on TTC39C mRNA in vitro. Cultured mouse primary hepatocytes at a density of 30,000 cells per well were transfected by electroporation with 8,000 nM antisense oligonucleotide. After a treatment period of approximately 24 hours, RNA was isolated from the cells and TTC39C mRNA levels were measured by quantitative real-time PCR. Mouse primer probe set RTS3266 (forward sequence AAGAAGGCTGAGCGATTTCG, designated herein as SEQ ID NO: 140; reverse sequence TCCACAAGTAGAGCACTTCAATGG, designated herein as SEQ ID NO: 141; probe sequence AAGCAAACCCCAACCAGAGCGCTG, designated herein as SEQ ID NO: 142). TTC39C mRNA levels were adjusted according to total RNA content, as measured by RIBOGREEN®. Results are presented as percent inhibition of TTC39C, relative to untreated control cells.

The chimeric antisense oligonucleotides targeting TTC39C were designed as 5-10-5 MOE gapmers. The gapmers are 20 nucleotides in length, wherein the central gap segment is comprised of ten 2′-deoxynucleotides and is flanked on both sides (in the 5′ and 3′ directions) by wings comprising five nucleotides each. Each nucleotide in the 5′ wing segment and each nucleotide in the 3′ wing segment has a 2′-MOE modification. The internucleoside linkages throughout each gapmer are phosphorothioate (P═S) linkages. All cytosine residues throughout each gapmer are 5-methylcytosines. “Mouse Target start site” indicates the 5′-most nucleotide to which the gapmer is targeted in the mouse gene sequence.

Over one hundred antisense oligonucleotides were designed to target at least one of the following murine TTC39C sequences: a mRNA sequence, designated herein as SEQ ID NO: 143 (GENBANK Accession No. NM_{—}028341.4), a genomic sequence, designated herein as SEQ ID NO: 144 (GENBANK Accession No. NT_{—}039674.7 truncated from nucleotides 9799001 to 9898000), SEQ ID NO: 145 (GENBANK Accession No. AK077971.1) or SEQ ID NO: 146 (GENBANK Accession No. AA511505.1). Table 29 shows an exemplary selection of thirty chimeric antisense oligonucleotides targeting TTC39C that exhibited good inhibition of TTC39C.

The murine oligonucleotides of Table 29 may also be cross-reactive with human mRNA sequence. ‘Mismatches’ indicate the number of nucleobases by which the murine oligonucleotide is mismatched with a human mRNA sequence. The greater the complementarity between the murine oligonucleotide and the human mRNA sequence, the more likely the murine oligonucleotide can cross-react with the human sequence. The murine oligonucleotides in Table 29 were compared to a human TTC39C sequence as shown in SEQ ID NO: 147 (GenBank Accession No. NM_{—}001135993.1). “Human Target start site” indicates the 5′-most nucleotide to which the antisense oligonucleotide is targeted in the human mRNA sequence.

TABLE 29
Inhibition of murine TTC39C mRNA levels by chimeric antisense oligonucleotides having 5-10-
5 MOE wings and deoxy gap targeted to various TTC39C sequences

| | Mouse | | | | | | |
|---|---|---|---|---|---|---|---|
| Mouse | Target | | | | Human | | SEQ |
| Start | SEQ | | | % | Start | | ID |
| Site | ID | ISIS No | Sequence | inhibition | Site | Mismatches | NO |
| 446 | 143 | 474592 | GAAGCATATTGATGCCAGCC | 93 | 554 | 3 | 148 |
| 465 | 143 | 474515 | TCCCTGAAGCCGTTGTTGAG | 96 | 573 | 0 | 149 |
| 509 | 143 | 474516 | TTAGTGGACTATGATTTCTG | 94 | 617 | 1 | 150 |
| 563 | 143 | 474517 | CCTCGAATGTCATCATGGCG | 98 | 671 | 2 | 151 |
| 592 | 143 | 474595 | GTCATCGCACGCCAACTGCA | 97 | 700 | 2 | 152 |
| 603 | 143 | 474518 | GTAGTCTTTAAGTCATCGCA | 96 | 711 | 3 | 153 |
| 641 | 143 | 474596 | CGATTACACCAGCCTCTTCA | 93 | 749 | 2 | 154 |
| 802 | 143 | 474598 | CCCACCTTTTATGTAGGCCG | 94 | n/a | n/a | 155 |
| 820 | 143 | 474521 | GGCTTTCCTAAGGATCCACC | 97 | 928 | 0 | 156 |
| 857 | 143 | 474599 | CGTTGATGTCCACATAGCAC | 92 | 965 | 3 | 157 |
| 902 | 143 | 474600 | CCAAGGGCTCTTCCGTCAGC | 90 | n/a | n/a | 158 |
| 949 | 143 | 474523 | GGTCACCCCTTCGGCCACAA | 98 | 1057 | 3 | 159 |
| 998 | 143 | 474601 | GGCCATATCCAAAGCTCACA | 91 | 1106 | 1 | 160 |
| 1202 | 143 | 474604 | TGTCACTGCCGTCCAAAGCA | 97 | 1310 | 3 | 161 |
| 1422 | 143 | 474607 | TCAATCATGCTGCACCAGCC | 98 | 1530 | 2 | 162 |
| 1496 | 143 | 474609 | CGTAGTAGCACTGGGACCAC | 95 | 1604 | 2 | 163 |
| 1550 | 143 | 474532 | GTTGTGCCCCATCCACATCA | 99 | 1658 | 2 | 164 |
| 1560 | 143 | 474610 | TTAAGAACAAGTTGTGCCCC | 90 | n/a | n/a | 165 |
| 1761 | 143 | 474535 | TCCACTTCATGGCAAGCTTG | 96 | 1869 | 0 | 166 |
| 1788 | 143 | 474613 | TGCTTTAATCCAACGACAGA | 90 | 1896 | 2 | 167 |
| 1894 | 143 | 474615 | GTACGAGTTACTCTGCCGAC | 96 | n/a | n/a | 168 |
| 1914 | 143 | 474538 | TAGCACGCATATGGCGGGAC | 96 | n/a | n/a | 169 |
| 2064 | 143 | 474617 | AGTTCCCTTAGGGAGGCCAG | 94 | 2172 | 3 | 170 |
| 2091 | 143 | 474540 | CCACTGGGAGTTTATCACTG | 98 | n/a | n/a | 171 |
| 2891 | 144 | 474543 | CCAGGTGGCTTTGGCTTGGG | 98 | n/a | n/a | 172 |
| 89499 | 144 | 474589 | TCCGAAATCGCTCAGCCTGC | 100 | 1745 | 3 | 173 |
| 98430 | 144 | 474654 | TCAGCTACAGAACGCAGGGT | n.d. | n/a | n/a | 174 |
| 49280 | 144 | 474662 | GGATTTCCGGGCATCAACCT | 95 | 796 | 3 | 175 |
| 84446 | 144 | 474666 | CAATCATGCTGCACCAGCCT | 95 | 1529 | 3 | 176 |
| 79 | 146 | 474629 | GATCACTGCCGTCCAAAGCA | 93 | 1310 | 3 | 177 |

### Example 9

### Dose-Dependent Antisense Inhibition of Murine TTC39C in Mouse Primary Hepatocytes

Thirty antisense oligonucleotides were selected from the study described in Example 8 and further tested at various doses in mouse primary hepatocytes. Cells were plated at a density of 30,000 cells per well and transfected using electroporation with 500 nM, 1,000 nM, 2,000 nM, 4,000 nM, and 8,000 nM concentrations of antisense oligonucleotide, as specified in Table 30. After a treatment period of approximately 16 hours, RNA was isolated from the cells and TTC39C mRNA levels were measured by quantitative real-time PCR. Murine primer probe set RTS3266 was used to measure mRNA levels. TTC39C mRNA levels were adjusted according to total RNA content, as measured by RIBOGREEN®. Results are presented as percent inhibition of TTC39C, relative to untreated control cells. As illustrated in Table 30, TTC39C mRNA levels were reduced in a dose-dependent manner in antisense oligonucleotide treated cells. ‘n/a’ indicates that the IC₅₀ for the particular antisense oligonucleotide could not be calculated in that dose range.

TABLE 30
Dose-dependent antisense inhibition of murine TTC39C mRNA
in mouse primary hepatocytes

| ISIS | 500.0 | | 2000.0 | | 8000.0 | IC₅₀ |
|---|---|---|---|---|---|---|
| No | nM | 1000.0 nM | nM | 4000.0 nM | nM | (μM) |
| 474515 | 56 | 62 | 79 | 91 | 94 | n/a |
| 474516 | 34 | 61 | 71 | 88 | 95 | 0.8 |
| 474517 | 60 | 67 | 89 | 95 | 97 | n/a |
| 474518 | 61 | 73 | 90 | 94 | 96 | n/a |
| 474521 | 46 | 63 | 84 | 92 | 97 | 0.5 |
| 474523 | 72 | 80 | 94 | 96 | 97 | n/a |
| 474532 | 51 | 64 | 84 | 94 | 97 | 0.4 |
| 474535 | 18 | 35 | 61 | 83 | 93 | 1.5 |
| 474538 | 51 | 58 | 82 | 88 | 92 | 0.4 |
| 474540 | 43 | 65 | 81 | 92 | 97 | 0.5 |
| 474543 | 77 | 88 | 88 | 88 | 94 | n/a |
| 474589 | 95 | 96 | 97 | 99 | 100 | n/a |
| 474592 | 24 | 45 | 71 | 84 | 92 | 1.2 |
| 474595 | 80 | 87 | 95 | 98 | 98 | n/a |
| 474596 | 47 | 65 | 62 | 90 | 96 | 0.6 |
| 474598 | 40 | 37 | 60 | 83 | 94 | 1.1 |
| 474599 | 36 | 59 | 60 | 84 | 92 | 0.9 |
| 474600 | 47 | 57 | 66 | 86 | 89 | 0.6 |
| 474601 | 36 | 49 | 65 | 87 | 92 | 1.0 |
| 474604 | 43 | 70 | 84 | 88 | 96 | 0.5 |
| 474607 | 40 | 65 | 85 | 92 | 94 | 0.5 |
| 474609 | 48 | 56 | 71 | 90 | 97 | 0.6 |
| 474610 | 10 | 28 | 38 | 65 | 71 | 2.8 |
| 474613 | 30 | 33 | 43 | 65 | 74 | 2.1 |
| 474615 | 33 | 43 | 82 | 92 | 98 | 0.9 |
| 474617 | 43 | 45 | 80 | 86 | 94 | 0.8 |
| 474629 | 16 | 28 | 42 | 48 | 66 | 3.6 |
| 474654 | 11 | 14 | 38 | 60 | 74 | 3.1 |
| 474662 | 28 | 35 | 65 | 88 | 94 | 1.3 |
| 474666 | 35 | 54 | 79 | 92 | 95 | 0.8 |

1. A method of reducing a tetratricopeptide repeat domain 39 isoform expression in an animal comprising administering to the animal a compound comprising a modified oligonucleotide 12 to 30 linked nucleosides in length targeted to the tetratricopeptide repeat domain 39 isoform, wherein expression of the tetratricopeptide repeat domain 39 is reduced in the animal.

2. A method of increasing HDL level and/or decreasing PCSK9 level in an animal comprising administering to the animal a compound comprising a modified oligonucleotide 12 to 30 linked nucleosides in length targeted to a tetratricopeptide repeat domain 39 isoform, wherein the modified oligonucleotide reduces the tetratricopeptide repeat domain 39 isoform expression in the animal, thereby increasing the HDL level in the animal.

3. (canceled)

4. A method for treating an animal with cardiovascular disease comprising
a. identifying said animal with cardiovascular disease,
b. administering to said animal a therapeutically effective amount of a compound comprising a modified oligonucleotide 12 to 30 linked nucleosides in length targeted to a tetratricopeptide repeat domain 39 isoform,
wherein said animal with cardiovascular disease is treated.

5. The method of claim 1, wherein the tetratricopeptide repeat domain 39 isoform is isoform A, B or C.

6. The method of claim 5, wherein the tetratricopeptide repeat domain 39A has the sequence shown in SEQ ID NO: 2, wherein the tetratricopeptide repeat domain 39B has the sequence shown in SEQ ID NO: 1 and wherein the tetratricopeptide repeat domain 39C has the sequence shown in SEQ ID NO: 3.

7.-8. (canceled)

9. A method of reducing tetratricopeptide repeat domain 39B expression, increasing HDL levels and/or decreasing PCSK9 levels comprising administering to an animal a compound comprising a modified oligonucleotide consisting of 12 to 30 linked nucleosides and having a nucleobase sequence at least 90% complementary to SEQ ID NO: 1 as measured over the entirety of said modified oligonucleotide, wherein expression of tetratricopeptide repeat domain 39B is reduced.

10.-14. (canceled)

15. The method of claim 4, wherein the therapeutically effective amount of the compound administered to the animal increases HDL and/or decreases PCSK9 in the animal.

16. (canceled)

17. The method of claim 1, wherein the modified oligonucleotide has a nucleobase sequence comprising at least 8 contiguous nucleobases of the nucleobase sequence recited in SEQ ID NO: 1, 2, or 3.

18. The method of claim 1, wherein the animal is a human.

19. The method of claim 1, wherein the compound is a first agent and further comprising administering a second agent.

20. The method of claim 19, wherein the first agent and the second agent are co-administered.

21. The method of claim 1, wherein administration comprises parenteral administration.

22. The method of claim 1, wherein the compound consists of a single-stranded modified oligonucleotide.

23. The method of claim 1, wherein the nucleobase sequence of the modified oligonucleotide is at least 95% or is 100% complementary to SEQ ID NO: 1, 2 or 3 as measured over the entirety of said modified oligonucleotide.

24. (canceled)

25. The method of claim 1, wherein at least one internucleoside linkage of said modified oligonucleotide is a modified internucleoside linkage, wherein at least one nucleoside of said modified oligonucleotide comprises a modified sugar and/or wherein at least one nucleoside of said modified oligonucleotide comprises a modified nucleobase.

26. The method of claim 25, wherein each internucleoside linkage is a phosphorothioate internucleoside linkage, wherein the modified nucleobase is a 5-methylcytosine, wherein at least one modified sugar is a bicyclic sugar and/or wherein at least one modified sugar comprises a 2′-O-methoxyethyl, methyl(methyleneoxy) (4′-CH(CH₃)—O-2′) BNA or a 4′-(CH₂)ₙ—O-2′ bridge, wherein n is 1 or 2.

27.-31. (canceled)

32. The method of claim 1, wherein the modified oligonucleotide comprises:
a. a gap segment consisting of linked deoxynucleosides;
b. a 5′ wing segment consisting of linked nucleosides;
c. a 3′ wing segment consisting of linked nucleosides;
wherein the gap segment is positioned between the 5′ wing segment and the 3′ wing segment and wherein each nucleoside of each wing segment comprises a modified sugar.

33. (canceled)

34. The method of claim 1, wherein the modified oligonucleotide consists of 20 linked nucleosides.

35. The method of claim 1, wherein the modified oligonucleotide comprises:
a. a gap segment consisting of ten linked deoxynucleosides;
b. a 5′ wing segment consisting of five linked nucleosides;
c. a 3′ wing segment consisting of five linked nucleosides;
wherein the gap segment is positioned between the 5′ wing segment and the 3′ wing segment, wherein each nucleoside of each wing segment comprises a 2′-O-methoxyethyl sugar, wherein each internucleoside linkage of said modified oligonucleotide is a phosphorothioate linkage, wherein each cytosine in said modified oligonucleotide is a 5′-methylcytosine and wherein said reduction of tetratricopeptide repeat domain 39B expression increases HDL in the animal.

36.-37. (canceled)

38. The method of claim 4, wherein the cardiovascular disease is arteriosclerosis, atherosclerosis, coronary heart disease, heart failure, hypertension, dyslipidemia, hypercholesterolemia, acute coronary syndrome, type II diabetes, type II diabetes with dyslipidemia, hepatic steatosis, non-alcoholic steatohepatitis, non-alcoholic fatty liver disease, hypertriglyceridemia, hyperfattyacidemia, hyperlipidemia and metabolic syndrome.

39.-41. (canceled)
